(19) United States
(12) Patent Application Publication
(10) Pub. No.: US 2024/0316167 A1
(43) Pub. Date: Sep. 26, 2024

(54) TREATMENT OF URTICARIA

(71) Applicant: EVAX AG, Münchwilen (CH)

(72) Inventor: Antonia GABRIEL, Münchwilen (CH)

(21) Appl. No.: 18/505,699

(22) Filed: Nov. 9, 2023

Related U.S. Application Data
(63) Continuation of application No. 17/057,438, filed on Nov. 20, 2020, now abandoned.

(30) Foreign Application Priority Data
May 28, 2018 (EP) 18174656.1
Nov. 12, 2018 (EP) 18205643.2

(51) Int. Cl. A61K 39/00; A61P 17/02; A61P 37/08

(52) U.S. Cl. CPC A61K 39/0005; A61P 17/02; A61P 37/08; A61K 2039/5258; A61K 2039/552

(57) ABSTRACT

The present invention relates to compositions, immunogenic or vaccine compositions and pharmaceutical compositions for the prevention or treatment of urticaria of equine mammals, preferably of horses. Furthermore, the invention provides methods for preventing or treating urticaria of equine mammals, preferably of horses.

### REFERENCE TO AN ELECTRONIC SEQUENCE LISTING

The contents of the electronic sequence listing (0192-0121US2.xml; Size: 23,282 bytes; and Date of Creation: Jan. 30, 2024) is herein incorporated by reference in its entirety.

The present invention relates to compositions, immunogenic or vaccine compositions and pharmaceutical compositions for the prevention or treatment of chronic recurrent urticaria of equine mammals, preferably of horses. Furthermore, the invention provides methods for preventing or treating urticaria of equine mammals, preferably of horses.

### RELATED ART

Recurrent (chronic) urticaria independent or associated with allergies is commonly seen in horses (Yu, AA AAEP Proceedings, Equine Dermatology. (2006) 52, 485-489). Recurrent urticaria in horses manifests in edematous wheals on the skin.

Clinical signs of urticaria in horses, also called true hives, are well-defined raised areas with lumps, wheals or rings and occur in the superficial dermis. In severe cases whole areas e.g. the face may become swollen. Urticaria pathogenesis comprises similar to type I allergies mast cell degranulation accompanied by basophil degranulation. Thus, chemical mediators such as histamine, heparin, cytokines, prostaglandins, leuktoriens and others lead to vascular permeability (angioedema) and inflammation causing wheal formation, the characteristic urticarial lesion (D W Scott, W H Miller, Skin immune system and allergic skin diseases (2003), 420-427; R R R Pascoe, D C Knottebbelt, Immune-mediated/allergic diseases, Manual of Equine Dermatology (1999), 156-160; C E Grattan, R A Sabroe, M W Greaves, Chronic urticaria, J Am Acad Dermatol (2002), 46(5): 645-647; S Rufenacht, E Marti, C von Tscharner, Immunoglobulin E-bearing cells and mast cells in skin biospies of horses with urticaria (2005), Vet Dermato 16(2):94-101; L Akucewich, G. Kunkle, Compendium Equine Edition (2007) 100-111; A. Diesel: Equine urticaria: a clinical guide to management; In Practice, (2014) Vol. 36, No. 6, 295-300). Such wheals and lesions, respectively, are usually a few millimeters to several centimeters in diameter and a few millimeters in height and pit with digital pressure. Urticaria and such wheals can appear as bizarre shapes and patterns and often coalesce to cover a large area and appear as a plaque. Lesions can appear anywhere; however, they are most common on the neck, trunk, and proximal extremities. While individual lesions are present for 24 to 48 hours, chronic urticaria episodes can last for at least six to eight weeks. Skin biopsies reveal a mild to moderate perivascular to interstitial dermatitis with numerous eosinophils and lymphocytes and variable dermal edema (D Q Scott, W H Miller, Equine Dermatology (2010), Skin immune system and allergic skin diseases, Chapter 8).

Causes of urticaria are various and include immunological causes and non-immunological causes. A challenge for long-term clinical management and cure is the identification of the underlying cause.

Immunological causes are, in particular, atopic dermatitis, food allergy, inhaled allergens, insect-bite hypersensitivity, in particular hypersensitivity reactions caused by insect-bites, vaccines and drugs (penicillin, tetracycline, sulfonamides, neomycin, ciprofloxacin, streptomycin, aspirin, phenylbutazone, flunixin, phenothiazines, guaifenesin, ivermectin, moxidectin, pethidine, iron, dextrans, hormones, vitamin B complex, and liver extracts), vasculitis, contact with a substance or material, infections (bacterial (e.g. strangles), viral (e.g. horse-pox), fungal, parasitic (e.g. Trypanosoma equi perdum), protozoal), snakebites.

Non-immunologic causes include dermatographism and pressure, cold temperature, heat, sunlight, psychological stress, exercise (L Akucewich, G. Kunkle, Compendium Equine Edition (2007) 100-111).

As indicated, the identification of the underlying cause and etiology is a challenge for long-term clinical management and cure, and treatment is often frustrating because recurrences are common (Yu, AA AAEP Proceedings, Equine Dermatology. (2006) 52, 485-489). Moreover, several contributing factors and underlying causes can manifest as urticarial lesions in the horse, identification of a specific trigger can be rather daunting for both the veterinary clinician and horse owner (A. Diesel: Equine urticaria: a clinical guide to management; In Practice, (2014) Vol. 36, No. 6, 295-300). Acute signs are often treated with systemic steroids although severe side effects might occur such osteoporosis and laminitis (Cunningham, F. M., et al. 2008, Vet. J. 177:334-344).

Thus, there is a need for prevention and treatment options of equine animals, in particular horses, which are affected by urticaria, in particular, by recurrent urticaria.

Recently, compositions comprising virus-like particles to which equine Il-5 antigens are attached have been described for the prevention or treatment of insect bite hypersensitivity (IBH), also known as “sweet itch” or “summer eczema”, of equine mammals, preferably of horses (WO2017/042212).

### SUMMARY OF THE INVENTION

It has surprisingly been found that the compositions of the present invention were able to prevent re-occurrence of urticaria episodes in horses chronically affected with urticaria. In detail, horses with yearly recurrent urticaria were vaccinated with preferred compositions of the present invention in the third year after a first year of no treatment and a second year of treatment with placebo. All horses developed urticaria hives in the untreated year and the placebo treated year, whereas all horses showed no clinical signs of urticaria in the third year vaccinated with preferred compositions of the present invention. Further, and importantly, a horse patient suffering from urticaria for approximately two years almost non-intermittently, and notably during all four seasons of the year was successfully vaccinated with preferred compositions of the present invention. Thus, following the second vaccination onwards, the horse was free of any clinical signs of urticaria. Thus, the compositions of the present invention are effective for the prevention and treatment of recurrent urticaria.

Thus, in a first aspect, the present invention provides for a composition comprising, preferably consisting of: (a) a core particle with at least one first attachment site; and (b) at least one antigen with at least one second attachment site, wherein said at least one antigen is an equine Interleukin-5 antigen (eIL-5 antigen), wherein said eIL-5 antigen comprises, or preferably is, a protein with the amino sequence selected from SEQ ID NO:1 or a protein with an amino acid sequence of at least 90%, preferably of at least 92%, further preferably of at least 95%, and again further preferably of at least 98% amino acid sequence identity with SEQ ID NO: 1; wherein (a) and (b) are linked through said at least one first and said at least one second attachment site via at least one non-peptide covalent bond; for use in a method of prevention or treatment of urticaria, preferably recurrent urticaria, of an equine mammal, preferably of a horse, wherein preferably an effective amount of said composition is administered to said equine mammal, preferably to said horse, and wherein said administration of said composition typically and preferably prevents or treats said urticarial, preferably said recurrent urticaria, in said equine mammal, preferably in said horse.

In a further aspect, the present invention provides for a pharmaceutical composition comprising said composition and a pharmaceutically acceptable carrier; for use in a method of prevention or treatment of urticaria, preferably recurrent urticaria, of an equine mammal, preferably of a horse, wherein preferably an effective amount of said pharmaceutical composition is administered to said equine mammal, preferably to said horse, and wherein said administration of said pharmaceutical composition typically and preferably prevents or treats said urticarial, preferably said recurrent urticaria, in said equine mammal, preferably in said horse.

In a further aspect, the present invention provides for a method of prevention or treatment of urticaria, preferably recurrent urticaria, of an equine mammal, preferably of a horse, wherein said method comprises administering an effective amount of the inventive composition or the inventive pharmaceutical composition to an equine mammal, preferably to a horse.

In another aspect, the present invention provides for the use of the inventive composition or said inventive pharmaceutical composition for the manufacture of a medicament for the prevention or treatment of urticaria, preferably recurrent urticaria, of an equine mammal, preferably of a horse, wherein typically and preferably an effective amount of said inventive composition or said inventive pharmaceutical composition is administered to an equine mammal, preferably to a horse.

Further aspects and embodiments of the present invention will become apparent as this description continues.

### DESCRIPTION OF FIGURES

FIG. 1A: Analysis of coupling reaction of eIL-5-C-His-CMVtt830. By SDS-PAGE. Proteins were stained with Coomassie blue: eIL-5 monomer (eIL-5, m), eIL-5 dimer (eIL-5, d), CMV (CMV, m), coupling (c). Lane M, Size Marker (See Blue, prestained, NuPAGE, Novex, Invitrogen Life Technologies), lane 1, TCEP activated eIL-5-C-His, lane 2, CMVtt830-VLP after derivatization with the chemical crosslinker SMPH, lane 3, eIL-5-C-His-CMVtt830coupling reaction.

FIG. 1B: Analysis of coupling reaction of eIL-5-C-His-CMVtt830. By Western-blot. Stained with α-His antibody: eIL-5 monomer (eIL-5, m), eIL-5 dimer (eIL-5, d), coupling (c). Lane M, Size Marker (See Blue, prestained, NuPAGE, Novex, Invitrogen Life Technologies), lane 1, TCEP activated eIL-5-C-His, lane 2, CMVtt830-VLP after derivatization with the chemical crosslinker SMPH, lane 3, eIL-5-C-His-CMVtt830 coupling reaction.

FIG. 2A: ELISA of anti-eIL-5 Antibody titer in horses. Sera from horses. Pre-immune and serum after 2^{nd} vaccination (several days after day 56) with eIL-5-C-His-Qβ vaccines of horse was collected. Sera were analyzed for antibodies against eIL-5. Four horses have been immunized on days 0, 28, 56 and 84 with eIL-5-C-His-Qβ. Data shows OD50 values for sera subtracted by pre-immune values.

FIG. 2B: ELISA of anti-Qβ Antibody titer in horses. Sera from horses. Pre-immune and serum after 2^{nd} vaccination (several days after day 56) with eIL-5-C-His-Qβ vaccines of horse was collected. Sera were analyzed for antibodies against Qβ. Four horses have been immunized on days 0, 28, 56 and 84 with eIL-5-C-His-Qβ. Data shows OD50 values for sera subtracted by pre-immune values.

FIG. 2C: ELISA of anti-eIL-5 Antibody titer in horses. Sera from horses. Pre-immune and serum after 2^{nd} vaccination (several days after day 56) with eIL-5-C-His-CMVtt830 vaccines of horse was collected. Sera were analyzed for antibodies against eIL-5. Thirteen horses have been immunized on days 0, 28, and 133 with eIL-5-C-His-CMVtt830. Data shows OD50 values for sera subtracted by pre-immune values.

FIG. 2D: ELISA of anti-CMVtt830 Antibody titer in horses. Sera from horses. Pre-immune and serum after 2^{nd} vaccination (several days after day 56) with eIL-5-C-His-CMVtt830 vaccines of horse was collected. Sera were analyzed for antibodies against CMVtt830. Thirteen horses have been immunized on days 0, 28, and 133 with eIL-5-C-His-CMVtt830. Data shows OD50 values for sera subtracted by pre-immune values.

FIG. 2E: ELISA of anti-eIL-5 Antibody titer in horses. Sera from horses. Pre-immune and serum after 2^{nd} vaccination (several days after day 56) with eIL-5-C-His-CMVtt830 vaccines of horse was collected. Sera were analyzed for antibodies against eIL-5. Three recurrent urticaria affected horses (horse 1, horse 2, and horse 3, see FIGS. 4 and 5) have been immunized on days 0, 28, and 133 with eIL-5-C-His-CMVtt830. Data shows OD50 values for sera subtracted by pre-immune values.

FIG. 2F: ELISA of anti-CMVtt830 Antibody titer in horses. Sera from horses. Pre-immune and serum after 2^{nd} vaccination (several days after day 56) with eIL-5-C-His-CMVtt830 vaccines of horse was collected. Sera were analyzed for antibodies against CMVtt830. Three recurrent urticaria horses (horse 1, horse 2, and horse 3; see FIGS. 4 and 5) have been immunized on days 0, 28, and 133 with eIL-5-C-His-CMVtt830. Data shows OD50 values for sera subtracted by pre-immune values.

FIG. 3: Reduction of eosinophil levels in blood upon generation of anti-eIL-5 antibodies by eIL-5-C-His-CMVtt830 vaccination. Eosinophil levels in blood were monitored in placebo-treated year (1) and eIL-5-C-His-CMVtt830 vaccinated year (2).

FIG. 4A: Urticaria Activity Score (UAS, y-axis) of horse 1 during year 1, no treatment (1), year 2, placebo treatment (2), year 3, eIL-5-C-His-CMVtt830 vaccination (3), and year 4, eIL-5-C-His-CMVtt830 vaccination (4).

FIG. 4B: Urticaria Activity Score (UAS, y-axis) of horse 2 during year 1, no treatment (1), year 2, placebo treatment (2), year 3, eIL-5-C-His-CMVtt830 vaccination (3), and year 4, eIL-5-C-His-CMVtt830 vaccination (4).

FIG. 4C: Urticaria Activity Score (UAS, y-axis) of horse 3 year 1, no treatment (2), year 2, eIL-5-C-His-CMVtt830 vaccination (3), and year 4, eIL-5-C-His-CMVtt830 vaccination (4).

FIG. 5A: Urticaria hives or healthy skin of horse 1 during year 2, placebo treatment (1), and year 3, eIL-5-C-His-CMVtt830 vaccination (2).

FIG. 5B: Urticaria hives or healthy skin of horse 3 during year 2, placebo treatment (1), and year 3, eIL-5-C-His-CMVtt830 vaccination (2).

FIG. 6A: Horse 4, untreated. Photographs of urticaria wheels before vaccination, in August 2017, October 2018 and December 2018.

FIG. 6B: Horse 4, vaccinated in January 2019 and February 2019. Photographs following the second vaccination of eIL-5-C-His-CMVtt830.

FIG. 6C: Urticaria Activity Score (UAS, y-axis) of horse 4 before vaccination (1), after two immunizations using eIL-5-C-His-CMVtt830 vaccination (2), and after three immunizations using eIL-5-C-His-CMVtt830 vaccination (3).

### DETAILED DESCRIPTION OF THE INVENTION

Unless defined otherwise, all technical and scientific terms used herein have the same meanings as commonly understood by one of ordinary skill in the art to which this invention belongs.

Virus-like particle (VLP): The term “virus-like particle (VLP)” as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. A virus-like particle in accordance with the invention is non-replicative and non-infectious since it lacks all or part of the viral genome or genome function. A virus-like particle in accordance with the invention may contain nucleic acid distinct from their genome. Recombinantly produced virus-like particles typically contain host cell derived RNA. A typical and preferred embodiment of a virus-like particle in accordance with the present invention is a viral capsid composed of polypeptides of the invention. A virus-like particle is typically a macromolecular assembly composed of viral coat protein which typically comprises 60, 120, 180, 240, 300, 360, or more than 360 protein subunits per virus-like particle. Typically and preferably, the interactions of these subunits lead to the formation of viral capsid or viral-capsid like structure with an inherent repetitive organization. One feature of a virus-like particle is its highly ordered and repetitive arrangement of its subunits.

Virus-like particle of an RNA bacteriophage: As used herein, the term “virus-like particle of an RNA bacteriophage” refers to a virus-like particle comprising, or preferably consisting essentially of or consisting of coat proteins, mutants or fragments thereof, of an RNA bacteriophage. In addition, virus-like particle of an RNA bacteriophage resembling the structure of an RNA bacteriophage, being non replicative and/or non-infectious, and lacking at least the gene or genes encoding for the replication machinery of the RNA bacteriophage, and typically also lacking the gene or genes encoding the protein or proteins responsible for viral attachment to or entry into the host. Also included are virus-like particles of RNA bacteriophages, in which the aforementioned gene or genes are still present but inactive, and, therefore, also leading to non-replicative and/or non-infectious virus-like particles of an RNA bacteriophage. Preferred VLPs derived from RNA bacteriophages exhibit icosahedral symmetry and consist of 180 subunits (monomers). Preferred methods to render a virus-like particle of an RNA bacteriophage non replicative and/or non-infectious is by physical, chemical inactivation, such as UV irradiation, formaldehyde treatment, typically and preferably by genetic manipulation.

Virus-like particle of CMV: The terms “virus-like particle of CMV” or CMV VLPs refer to a virus-like particle comprising, or preferably consisting essentially of, or preferably consisting of at least one CMV polypeptide. Preferably, a virus-like particle of CMV comprises said CMV polypeptide as the major, and even more preferably as the sole protein component of the capsid structure. Typically and preferably, virus-like particles of CMV resemble the structure of the capsid of CMV. Virus-like particles of CMV are non-replicative and/or non-infectious, and lack at least the gene or genes encoding for the replication machinery of the CMV, and typically also lack the gene or genes encoding the protein or proteins responsible for viral attachment to or entry into the host. This definition includes also virus-like particles in which the aforementioned gene or genes are still present but inactive. Preferred methods to render a virus-like particle of CMV non replicative and/or non-infectious is by physical or chemical inactivation, such as UV irradiation, formaldehyde treatment. Preferably, VLPs of CMV lack the gene or genes encoding for the replication machinery of the CMV, and also lack the gene or genes encoding the protein or proteins responsible for viral attachment to or entry into the host. Again more preferably, non-replicative and/or non-infectious virus-like particles are obtained by recombinant gene technology. Recombinantly produced virus-like particles of CMV according to the invention typically and preferably do not comprise the viral genome. Virus-like particles comprising more than one species of polypeptides, often referred to as mosaic VLPs are also encompassed by the invention. Thus, in one embodiment, the virus-like particle according to the invention comprises at least two different species of polypeptides, wherein at least one of said species of polypeptides is a CMV polypeptide. Preferably, a VLP of CMV is a macromolecular assembly composed of CMV coat protein which typically comprises 180 coat protein subunits per VLP. Typically and preferably, a VLP of CMV as used herein, comprises, essentially consists of, or alternatively consists of, at least one CMV polypeptide comprising or preferably consisting of (i) an amino acid sequence of a coat protein of CMV; or (ii) a mutated amino acid sequence, wherein the amino acid sequence to be mutated is an amino acid sequence of a coat protein of CMV, and wherein said mutated amino acid sequence and said amino acid sequence to be mutated show a sequence identity of at least 90%, preferably of at least 95%, further preferably of at least 98% and again more preferably of at least 99%.

Antigen: As used herein, the term “antigen” refers to a molecule capable of being bound by an antibody or a T-cell receptor (TCR) if presented by MHC molecules. The term “antigen”, as used herein, also refers to T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and/or is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. If not indicated otherwise, the term “antigen” as used herein does not refer to the core particle or virus-like particle contained in the inventive compositions, immunogenic or vaccine compositions and/or pharmaceutical compositions.

Coat protein: The term “coat protein” refers to a viral protein, preferably to a subunit of a natural capsid of a virus, preferably of an RNA bacteriophage or a plant virus, which is capable of being incorporated into a virus capsid or a VLP. The term coat protein encompasses naturally occurring coat protein as well as recombinantly expressed coat protein. Further encompassed are mutants and fragments of coat protein, wherein said mutants and fragments retains the capability of forming a VLP.

Polypeptide: The term “polypeptide” as used herein refers to a polymer composed of amino acid monomers which are linearly linked by peptide bonds (also known as amide bonds). The term polypeptide refers to a consecutive chain of amino acids and does not refer to a specific length of the product. Thus, peptides, and proteins are included within the definition of polypeptide.

Cucumber Mosaic Virus (CMV) polypeptide: The term “cucumber mosaic virus (CMV) polypeptide” as used herein refers to a polypeptide comprising or preferably consisting of: (i) an amino acid sequence of a coat protein of cucumber mosaic virus (CMV), or (ii) a mutated amino acid sequence, wherein the amino acid sequence to be mutated is an amino acid sequence of a coat protein of CMV, and wherein said mutated amino acid sequence and said amino acid sequence to be mutated, i.e. said coat protein of CMV, show a sequence identity of at least 90%, preferably of at least 95%, further preferably of at least 98% and again more preferably of at least 99%. Typically and preferably, the CMV polypeptide is capable of forming a virus-like particle of CMV upon expression by self-assembly.

Coat protein (CP) of cucumber mosaic virus (CMV): The term “coat protein (CP) of cucumber mosaic virus (CMV)”, as used herein, refers to a coat protein of the cucumber mosaic virus which occurs in nature. Due to extremely wide host range of the cucumber mosaic virus, a lot of different strains and isolates of CMV are known and the sequences of the coat proteins of said strains and isolates have been determined and are, thus, known to the skilled person in the art as well. The sequences of said coat proteins (CPs) of CMV are described in and retrievable from the known databases such as Genbank, www.dpvweb.net, or www.ncbi.nlm.nih.gov/protein/. Examples are described in EP Application No. 14189897.3. Further examples of CMV coat proteins are provided in SEQ ID NOs 15-17. It is noteworthy that these strains and isolates have highly similar coat protein sequences at different protein domains, including the N-terminus of the coat protein. In particular, 98.1% of all completely sequenced CMV isolates share more than 85% sequence identity within the first 28 amino acids of their coat protein sequence, and still 79.5% of all completely sequenced CMV isolates share more than 90% sequence identity within the first 28 amino acids of their coat protein sequence.

Typically and preferably, the coat protein of CMV used for the present invention is capable of forming a virus-like particle of CMV upon expression by self-assembly. Preferably, the coat protein of CMV used for the present invention is capable of forming a virus-like particle of CMV upon expression by self-assembly in E. coli.

Modified virus-like particle (VLP) of cucumber mosaic virus (CMV): The term “modified virus-like particle (VLP) of cucumber mosaic virus (CMV)” as used herein, refers to a VLP of CMV which is a modified one in such as it comprises, or preferably consists essentially of, or preferably consists of at least one modified CMV polypeptide, wherein said modified CMV polypeptide comprises, or preferably consists of, a CMV polypeptide, and a T helper cell epitope. Typically and preferably, said T helper cell epitope (i) is fused to the N-terminus of said CMV polypeptide, (ii) is fused to the C-terminus of said CMV polypeptide, (iii) replaces a region of consecutive amino acids of said CMV polypeptide, wherein the sequence identity between said replaced region of consecutive amino acids of said CMV polypeptide and the T helper cell epitope is at least 15%, preferably at least 20%, or (iv) replaces a N-terminal region of said CMV polypeptide, and wherein said replaced N-terminal region of said CMV polypeptide consists of 5 to 15 consecutive amino acids. Preferably, said T helper cell epitope replaces a N-terminal region of said CMV polypeptide, and wherein said replaced N-terminal region of said CMV polypeptide consists of 5 to 15 consecutive amino acids, preferably of 9 to 14 consecutive amino acids, more preferably of 11 to 13 consecutive amino acids, and most preferably of 11, 12 or 13 consecutive amino acids. Preferably said modified VLP of CMV of the present invention is a recombinant modified VLP of CMV.

Modified CMV polypeptide: The term “modified CMV polypeptide” as used herein refers to a CMV polypeptide modified in such as defined herein, that said modified CMV polypeptide comprises, or preferably consists of, a CMV polypeptide, and a T helper cell epitope. Typically, the modified CMV polypeptide is capable of forming a virus-like particle of CMV upon expression by self-assembly. Preferably, the modified CMV polypeptide is a recombinant modified CMV polypeptide and is capable of forming a virus-like particle of CMV upon expression by self-assembly in E. coli.

N-terminal region of the CMV polypeptide: The term “N-terminal region of the CMV polypeptide” as used herein, refers either to the N-terminus of said CMV polypeptide, and in particular to the N-terminus of a coat protein of CMV, or to the region of the N-terminus of said CMV polypeptide or said coat protein of CMV but starting with the second amino acid of the N-terminus of said CMV polypeptide or said coat protein of CMV if said CMV polypeptide or said coat protein comprises a N-terminal methionine residue. Preferably, in case said CMV polypeptide or said coat protein comprises a N-terminal methionine residue, from a practical point of view, the start-codon encoding methionine will usually be deleted and added to the N-terminus of the Th cell epitope. Further preferably, one, two or three additional amino acids, preferably one amino acid, may be optionally inserted between the stating methionine and the Th cell epitope for cloning purposes. The term “N-terminal region of the mutated amino acid sequence of a CMV polypeptide or a CMV coat protein” as used herein, refers either to the N-terminus of said mutated amino acid sequence of said CMV polypeptide or said coat protein of CMV, or to the region of the N-terminus of said mutated amino acid sequence of said CMV polypeptide or said coat protein of CMV but starting with the second amino acid of the N-terminus of said mutated amino acid sequence of said CMV polypeptide or said coat protein of CMV if said mutated amino acid sequence comprises a N-terminal methionine residue. Preferably, in case said CMV polypeptide or said coat protein comprises a N-terminal methionine residue, from a practical point of view, the start-codon encoding methionine will usually be deleted and added to the N-terminus of the Th cell epitope. Further preferably, one, two or three additional amino acids, preferably one amino acid, may be optionally inserted between the stating methionine and the Th cell epitope for cloning purposes.

Recombinant polypeptide: In the context of the invention the term “recombinant polypeptide” refers to a polypeptide which is obtained by a process which comprises at least one step of recombinant DNA technology. Typically and preferably, a recombinant polypeptide is produced in a prokaryotic expression system. It is apparent for the artisan that recombinantly produced polypeptides which are expressed in a prokaryotic expression system such as E. coli may comprise an N-terminal methionine residue. The N-terminal methionine residue is typically cleaved off the recombinant polypeptide in the expression host during the maturation of the recombinant polypeptide. However, the cleavage of the N-terminal methionine may be incomplete. Thus, a preparation of a recombinant polypeptide may comprise a mixture of otherwise identical polypeptides with and without an N-terminal methionine residue. Typically and preferably, a preparation of a recombinant polypeptide comprises less than 10%, more preferably less than 5%, and still more preferably less than 1% recombinant polypeptide with an N-terminal methionine residue.

Recombinant CMV polypeptide: The term “recombinant CMV polypeptide” refers to a CMV polypeptide as defined above which is obtained by a process which comprises at least one step of recombinant DNA technology. Typically and preferably a preparation of a recombinant CMV polypeptide comprises less than 10%, more preferably less than 5%, and still more preferably less than 1% recombinant CMV polypeptide with an N-terminal methionine residue. Consequently, a recombinant virus-like particle of the invention may comprise otherwise identical recombinant polypeptides with and without an N-terminal methionine residue.

Recombinant modified CMV polypeptide: The term “recombinant modified CMV polypeptide” refers to a modified CMV polypeptide as defined above which is obtained by a process which comprises at least one step of recombinant DNA technology. Typically and preferably a preparation of a recombinant modified CMV polypeptide comprises less than 10%, more preferably less than 5%, and still more preferably less than 1% recombinant modified CMV polypeptide with an N-terminal methionine residue. Consequently, a recombinant virus-like particle of the invention may comprise otherwise identical recombinant polypeptides with and without an N-terminal methionine residue.

Recombinant virus-like particle: In the context of the invention the term “recombinant virus-like particle” refers to a virus-like particle (VLP) which is obtained by a process which comprises at least one step of recombinant DNA technology. Typically and preferably a recombinant VLP is obtained by expression of a recombinant viral coat protein in host, preferably in a bacterial cell. Typically and preferably, a recombinant virus-like particle comprises at least one recombinant polypeptide, preferably a recombinant CMV polypeptide or recombinant modified CMV polypeptide. Most preferably, a recombinant virus-like particle is composed of or consists of recombinant CMV polypeptides or recombinant modified CMV polypeptides. As a consequence, if in the context of the present invention the definition of inventive recombinant VLPs are effected with reference to specific amino acid sequences comprising a N-terminal methionine residue the scope of these inventive recombinant VLPs encompass the VLPs formed by said specific amino acid sequences without said N-terminal methionine residue but as well, even though typically in a minor amount as indicated herein, the VLPs formed by said specific amino acid sequences with said N-terminal methionine. Furthermore, it is within the scope of the present invention that if the definition of inventive recombinant VLPs are effected with reference to specific amino acid sequences comprising a N-terminal methionine residue VLPs are encompassed comprising both amino acid sequences comprising still said N-terminal methionine residue and amino acid sequences lacking the N-terminal methionine residue.

Mutated amino acid sequence: The term “mutated amino acid sequence” refers to an amino acid sequence which is obtained by introducing a defined set of mutations into an amino acid sequence to be mutated. In the context of the invention, said amino acid sequence to be mutated typically and preferably is an amino acid sequence of a coat protein of CMV. Thus, a mutated amino acid sequence differs from an amino acid sequence of a coat protein of CMV in at least one amino acid residue, wherein said mutated amino acid sequence and said amino acid sequence to be mutated show a sequence identity of at least 90%. Typically and preferably said mutated amino acid sequence and said amino acid sequence to be mutated show a sequence identity of at least 91%, 92%, 93% 94%, 95%, 96%, 97%, 98%, or 99%. Preferably, said mutated amino acid sequence and said sequence to be mutated differ in at most 11, 10, 9, 8, 7, 6, 4, 3, 2, or 1 amino acid residues, wherein further preferably said difference is selected from insertion, deletion and amino acid exchange. Preferably, the mutated amino acid sequence differs from an amino acid sequence of a coat protein of CMV in least one amino acid, wherein preferably said difference is an amino acid exchange.

Position corresponding to residues . . . : The position on an amino acid sequence, which is corresponding to given residues of another amino acid sequence can be identified by sequence alignment, typically and preferably by using the BLASTP algorithm, most preferably using the standard settings. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Sequence identity: The sequence identity of two given amino acid sequences is determined based on an alignment of both sequences. Algorithms for the determination of sequence identity are available to the artisan. Preferably, the sequence identity of two amino acid sequences is determined using publicly available computer homology programs such as the “BLAST” program (http://blast.ncbi.nlm.nih.gov/Blast.cgi) or the “CLUSTALW” (http://www.genome.jp/tools/clustalw/), and hereby preferably by the “BLAST” program provided on the NCBI homepage at http://blast.ncbi.nlm.nih.gov/Blast.cgi, using the default settings provided therein. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Amino acid exchange: The term amino acid exchange refers to the exchange of a given amino acid residue in an amino acid sequence by any other amino acid residue having a different chemical structure, preferably by another proteinogenic amino acid residue. Thus, in contrast to insertion or deletion of an amino acid, the amino acid exchange does not change the total number of amino acids of said amino acid sequence. Very preferred in the context of the invention is the exchange of an amino acid residue of said amino acid sequence to be mutated by a lysine residue or by a cysteine residue.

Epitope: The term epitope refers to continuous or discontinuous portions of an antigen, preferably a polypeptide, wherein said portions can be specifically bound by an antibody or by a T-cell receptor within the context of an MHC molecule. With respect to antibodies, specific binding excludes non-specific binding but does not necessarily exclude cross-reactivity. An epitope typically comprise 5-20 amino acids in a spatial conformation which is unique to the antigenic site.

T helper (Th) cell epitope: The term “T helper (Th) cell epitope” as used herein refers to an epitope that is capable of recognition by a helper Th cell. In another preferred embodiment, said T helper cell epitope is a universal T helper cell epitope.

Universal Th cell epitope: The term “universal Th cell epitope” as used herein refers to a Th cell epitope that is capable of binding to at least one, preferably more than one MHC class II molecules. The simplest way to determine whether a peptide sequence is a universal Th cell epitope is to measure the ability of the peptide to bind to individual MHC class II molecules. This may be measured by the ability of the peptide to compete with the binding of a known Th cell epitope peptide to the MHC class II molecule. A representative selection of HLA-DR molecules are described in e.g. Alexander J, et al., Immunity (1994) 1:751-761. Affinities of Th cell epitopes for MHC class II molecules should be at least 10⁻⁵M. An alternative, more tedious but also more relevant way to determine the “universality” of a Th cell epitope is the demonstration that a larger fraction of people (>30%) generate a measurable T cell response upon immunization and boosting one months later with a protein containing the Th cell epitope formulated in IFA. A representative collection of MHC class II molecules present in different individuals is given in Panina-Bordignon P, et al., Eur J Immunol (1989) 19:2237-2242. As a consequence, the term “universal Th cell epitope” as used herein preferably refers to a Th cell epitope that generates a measurable T cell response upon immunization and boosting (one months later with a protein containing the Th cell epitope formulated in IFA) in more than 30% of a selected group of individuals as described in Panina-Bordignon P, et al., Eur J Immunol (1989) 19:2237-2242. Moreover, and again further preferred, the term “universal Th cell epitope” as used herein preferably refers to a Th cell epitope that is capable of binding to at least one, preferably to at least two, and even more preferably to at least three DR alleles selected from of DR1, DR2w2b, DR3, DR4w4, DR4w14, DR5, DR7, DR52a, DRw53, DR2w2a; and preferably selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40. In an even again more preferable manner, the term “universal Th cell epitope” as used herein refers to a Th cell epitope that is capable of binding to at least one, preferably to at least two, and even more preferably to at least three DR alleles selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40.

Universal Th cell epitopes are described, and known to the skilled person in the art, such as by Alexander J, et al., Immunity (1994) 1:751-761, Panina-Bordignon P, et al., Eur J Immunol (1989) 19:2237-2242, Calvo-Calle J M, et al., J Immunol (1997) 159:1362-1373, and Valmori D, et al., J Immunol (1992) 149:717-721.

Adjuvant: The term “adjuvant” as used herein refers to non-specific stimulators of the immune response or substances that allow generation of a depot in the host which when combined with the vaccine and pharmaceutical composition, respectively, of the present invention may provide for an even more enhanced immune response. Preferred adjuvants are complete and incomplete Freund's adjuvant, aluminum containing adjuvant, preferably aluminum hydroxide, and modified muramyldipeptide. Further preferred adjuvants are mineral gels such as aluminum hydroxide, surface active substances such as lyso lecithin, pluronic polyols, polyanions, peptides, oil emulsions, keyhole limpet hemocyanins, dinitrophenol, and human adjuvants such as BCG (bacille Calmette Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminum salts (Alum), MF-59, OM-174, OM-197, OM-294, and Virosomal adjuvant technology. The adjuvants may also comprise mixtures of these substances. Virus-like particles have been generally described as an adjuvant. However, the term “adjuvant”, as used within the context of this application, refers to an adjuvant not being the inventive virus-like particle. Rather “adjuvant” relates to an additional, distinct component of the inventive compositions, vaccines or pharmaceutical compositions.

Effective amount: As used herein, the term “effective amount” refers to an amount of an active ingredient, typically and preferably a composition in accordance with the present invention, sufficient to effect beneficial or desired results when administered to an equine mammal, preferably to a horse. An effective amount can be administered in one or more administrations, applications or dosages. An effective amount of the composition, or alternatively the pharmaceutical composition, would be the amount that achieves this selected result, and such an amount could be determined as a matter of routine by a person skilled in the art. Preferably, the term “effective amount”, as used herein, refers to an amount that produces an objectively measured change in one or more parameter associated with the prevention or treatment of urticaria, preferably recurrent urticaria, of an equine mammal, preferably of a horse. Again further preferably, said one or more parameter associated with the prevention or treatment of urticaria, preferably recurrent urticaria, of an equine mammal, preferably of a horse is the level or severity grade of the urticaria by area of hives. Again further preferably, said reduction of said level or severity grade of the urticaria by area of hives is determined by a urticaria activity scoring test. The effective amount can vary depending upon the particular equine mammal, preferably the horse, and condition being treated, the weight and age of the equine mammal, preferably the horse, the severity of the disease or symptom condition, the particular composition chosen, the dosing regimen to be followed, timing of administration, the manner of administration and the like, all of which can readily be determined by one of ordinary skill in the art without necessitating undue experimentation.

Treatment: As used herein, the terms “treatment”, “treat”, “treated” or “treating” refer to prophylaxis and/or therapy. In one embodiment, the terms “treatment”, “treat”, “treated” or “treating” refer to a therapeutic treatment. In another embodiment, the terms “treatment”, “treat”, “treated” or “treating” refer to a prophylactic treatment. Typically and preferably, equine mammals, preferably horses, in need of treatment include those already with the disorder as well as those in which the disorder is to be prevented. Thus, preferably, the terms “treatment”, “treat”, “treated” or “treating” of a disease, condition or disorder in accordance with the present invention, includes preventing or protecting against the disease, condition or disorder (that is, causing the symptoms not to develop); inhibiting the disease, condition or disorder (i.e., arresting or suppressing the development of symptoms; and/or relieving the disease, condition or disorder (i.e., causing the regression of symptoms). As will be appreciated, it is not always possible to distinguish between “preventing” and “suppressing” a disease, condition or disorder since the ultimate inductive event or events may be unknown or latent. Accordingly, the term “prophylaxis” will be understood to constitute a type of “treatment” that encompasses both “preventing” and “suppressing.” The term “treatment” thus includes “prophylaxis”.

The term “prophylaxis” as used herein refers to means of preventing or delaying the onset of disease or condition and/or symptoms attributed to the disease or condition.

Attachment Site, First: As used herein, the phrase “first attachment site” refers to an element which is naturally occurring with the virus-like particle or which is artificially added to the virus-like particle, and to which the second attachment site may be linked. The first attachment site preferably is a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the first attachment site is the amino group of an amino acid residue, preferably of a lysine residue. The first attachment site is typically located on the surface, and preferably on the outer surface of the VLP. Multiple first attachment sites are present on the surface, preferably on the outer surface of the VLP, typically in a repetitive configuration. In a preferred embodiment the first attachment site is associated with the VLP, through at least one covalent bond, preferably through at least one peptide bond. In a further preferred embodiment the first attachment site is naturally occurring with the VLP. Alternatively, in a preferred embodiment the first attachment site is artificially added to the VLP. In a very preferred embodiment said first attachment site is the amino group of a lysine residue of the amino acid sequence of said VLP polypeptide.

Attachment Site, Second: As used herein, the phrase “second attachment site” refers to an element which is naturally occurring with or which is artificially added to the antigen and to which the first attachment site may be linked. The second attachment site of the antigen preferably is a protein, a polypeptide, a peptide, an amino acid, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the second attachment site is a sulfhydryl group, preferably the sulfhydryl group of the amino acid cysteine most preferably the sulfhydryl group of a cysteine residue. The term “antigen with at least one second attachment site” refers, therefore, to a construct comprising the antigen and at least one second attachment site. However, in particular for a second attachment site, which is not naturally occurring within the antigen, such a construct typically and preferably further comprises a “linker”. In another preferred embodiment the second attachment site is associated with the antigen through at least one covalent bond, preferably through at least one peptide bond. In a further embodiment, the second attachment site is naturally occurring within the antigen. In another further preferred embodiment, the second attachment site is artificially added to the antigen through a linker, wherein said linker comprises or alternatively consists of a cysteine. Preferably, the linker is fused to the antigen by a peptide bond.

Linked: The terms “linked” or “linkage” as used herein, refer to all possible ways, preferably chemical interactions, by which the at least one first attachment site and the at least one second attachment site are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, carbon-phosphorus bonds, carbon-sulfur bonds such as thioether, or imide bonds. In certain preferred embodiments the first attachment site and the second attachment site are linked through at least one covalent bond, preferably through at least one non-peptide bond, and even more preferably through exclusively non-peptide bond(s). The term “linked” as used herein, however, shall not only refer to a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker. In other preferred embodiments the first attachment site and the second attachment site are linked through at least one covalent bond, preferably through at least one peptide bond, and even more preferably through exclusively peptide bond(s).

Linker: A “linker”, as used herein, either associates the second attachment site with the antigen or already comprises, essentially consists of, or consists of the second attachment site. Preferably, a “linker”, as used herein, already comprises the second attachment site, typically and preferably—but not necessarily—as one amino acid residue, preferably as a cysteine residue. A preferred linkers are an amino acid linkers, i.e. linkers containing at least one amino acid residue. The term amino acid linker does not imply that such a linker consists exclusively of amino acid residues. However, a linker consisting exclusively of amino acid residues is a preferred embodiment of the invention. The amino acid residues of the linker are, preferably, composed of naturally occurring amino acids or unnatural amino acids known in the art, all-L or all-D or mixtures thereof. Further preferred embodiments of a linker in accordance with this invention are molecules comprising a sulfhydryl group or a cysteine residue and such molecules are, therefore, also encompassed within this invention. Association of the linker with the antigen is preferably by way of at least one covalent bond, more preferably by way of at least one peptide bond.

Equine mammal: An “equine mammal”, as used herein, is a mammal included in the family Equidae including horses, ponys, asses (donkeys), and zebras. Preferably, the term “equine mammal”, as used herein, refers to a horse, a pony, an ass (a donkey), and a zebra. Again more preferably, the term “equine mammal”, as used herein, refers to a horse.

Several aspects of the present invention are disclosed herein; the embodiments and preferred embodiments, respectively, mentioned further herein are applicable for each and any aspect of the present invention disclosed herein, even though not explicitly mentioned.

The compositions of the present invention were able to prevent re-occurrence of urticaria episodes in horses chronically affected with urticaria. Horses with yearly recurrent urticaria were vaccinated with preferred compositions of the present invention in the third year after a first year of no treatment and a second year of treatment with placebo. All horses developed urticaria hives in the untreated year and the placebo treated year, whereas all horses showed no clinical signs of urticaria in the third year vaccinated with preferred compositions of the present invention. Thus, the compositions of the present invention are effective for the prevention and treatment of recurrent urticaria.

Thus, in a first aspect, the present invention provides for a composition comprising, preferably consisting of: (a) a core particle with at least one first attachment site; and (b) at least one antigen with at least one second attachment site, wherein said at least one antigen is an equine Interleukin-5 antigen (eIL-5 antigen), wherein said eIL-5 antigen comprises, or preferably is, a protein with the amino sequence selected from SEQ ID NO:1 or a protein with an amino acid sequence of at least 90%, preferably of at least 92%, further preferably of at least 95%, and again further preferably of at least 98% amino acid sequence identity with SEQ ID NO: 1; wherein (a) and (b) are linked through said at least one first and said at least one second attachment site via at least one non-peptide covalent bond; for use in a method of prevention or treatment of urticaria, preferably recurrent urticaria, of an equine mammal, preferably of a horse, wherein preferably an effective amount of said composition is administered to said equine mammal, preferably to said horse, and wherein said administration of said composition typically and preferably prevents or treats said urticarial, preferably said recurrent urticaria, in said equine mammal, preferably in said horse.

In a further aspect, the present invention provides for a pharmaceutical composition comprising said composition and a pharmaceutically acceptable carrier; for use in a method of prevention or treatment of urticaria, preferably recurrent urticaria, of an equine mammal, preferably of a horse, wherein preferably an effective amount of said pharmaceutical composition is administered to said equine mammal, preferably to said horse, and wherein said administration of said pharmaceutical composition typically and preferably prevents or treats said urticarial, preferably said recurrent urticaria, in said equine mammal, preferably in said horse.

In a further aspect, the present invention provides for a method of prevention or treatment of urticaria, preferably recurrent urticaria, of an equine mammal, preferably of a horse, wherein said method comprises administering an effective amount of the inventive composition or the inventive pharmaceutical composition to an equine mammal, preferably to a horse.

In a preferred embodiment, said prevention or treatment of urticaria is not the prevention or treatment of insect bite hypersensitivity (IBH) of an equine mammal, preferably of a horse. In another preferred embodiment, said prevention or treatment of urticaria is not the prevention or treatment of urticaria caused by insect bite hypersensitivity (IBH) of an equine mammal, preferably of a horse. In another preferred embodiment, said prevention or treatment of urticaria is not the prevention or treatment of urticaria induced by insect bite hypersensitivity (IBH) of an equine mammal, preferably of a horse. In another preferred embodiment, said prevention or treatment of urticaria is not caused by hypersensitivity reactions caused by insect bites. In another preferred embodiment, said prevention or treatment of urticaria is not induced by hypersensitivity reactions caused by insect bites.

In another preferred embodiment, said method is a method of prevention of urticaria, preferably recurrent urticaria, of an equine mammal, preferably of a horse.

In another preferred embodiment, said eIL-5 antigen comprises, or preferably is, a protein with the amino sequence selected from SEQ ID NO:1, SEQ ID NO:2, SEQ ID NO:3, SEQ ID NO:4 and SEQ ID NO:5. In a further preferred embodiment, said eIL-5 antigen comprises, or preferably is, a protein with the amino sequence of SEQ ID NO:1. In a further preferred embodiment, said eIL-5 antigen comprises, or preferably is, a protein with the amino sequence of SEQ ID NO:2. In a further preferred embodiment, said eIL-5 antigen comprises, or preferably is, a protein with the amino sequence of SEQ ID NO:3. In a further preferred embodiment, said eIL-5 antigen comprises, or preferably is, a protein with the amino sequence of SEQ ID NO:4. In a further preferred embodiment, said eIL-5 antigen comprises, or preferably is, a protein with the amino sequence of SEQ ID NO:5.

In another preferred embodiment, said composition for use does not comprise an equine Interleukin-31 antigen (eIL-31 antigen). In a further preferred embodiment, said composition for use does not comprise an equine Eotaxin antigen (eEotaxin antigen). In another preferred embodiment, said composition for use does not comprise an eIL-31 antigen or an eEotaxin antigen. In another preferred embodiment, said composition for use does not comprise an eIL-31 antigen and an eEotaxin antigen. In another preferred embodiment, said composition for use does not comprise an eIL-31 antigen, wherein said eIL-31 antigen comprises, or preferably is, a protein with the amino sequence selected from SEQ ID NO:13 or a protein with an amino acid sequence of at least 90%, preferably of at least 92%, further preferably of at least 95%, and again further preferably of at least 98% amino acid sequence identity with SEQ ID NO:13. In a further preferred embodiment, said at least one eIL-5 antigen linked to said core particle is the sole active ingredient of said composition for said prevention or treatment of urticaria.

In another preferred embodiment, said method does not comprise administering of a composition comprising an eIL-31 antigen to said equine mammal, preferably to said horse. In a further preferred embodiment, said method does not comprise administering of a composition comprising an eEotaxin antigen. In another preferred embodiment, said method does not comprise administering of a composition comprising an eIL-31 antigen or an eEotaxin antigen. In another preferred embodiment, said method does not comprise administering of a composition comprising an eIL-31 antigen and an eEotaxin antigen. In another preferred embodiment, said method does not comprise administering of a composition comprising an eIL-31 antigen, wherein said eIL-31 antigen comprises, or preferably is, a protein with the amino sequence selected from SEQ ID NO:13 or a protein with an amino acid sequence of at least 90%, preferably of at least 92%, further preferably of at least 95%, and again further preferably of at least 98% amino acid sequence identity with SEQ ID NO:13.

In a further preferred embodiment, said method does not comprise a combination treatment of said equine mammal, preferably to said horse, of administering of at least two different compositions, one of said at least two different compositions comprising an eIL-5 antigen and an another of said at least two different compositions comprising an eIL-31 antigen.

In a further preferred embodiment, said method does not comprise a combinatory treatment of said equine mammal, preferably to said horse, of administering of at least two different compositions, one of said at least two different compositions comprising an eIL-5 antigen and an another of said at least two different compositions comprising an eIL-31 antigen, wherein said combinatory treatment is an administration of said at least two different compositions at the same or at a different time and/or at the same or at a different administration or injection site.

In a further preferred embodiment, said method does not comprise administering of an eIL-31 antigen to said equine mammal, preferably to said horse. In another preferred embodiment, said method does not comprise administering of an eEotaxin antigen. In another preferred embodiment, said method does not comprise administering of an eIL-31 antigen or an eEotaxin antigen. In another preferred embodiment, said method does not comprise administering of an eIL-31 antigen and an eEotaxin antigen. In another preferred embodiment, said method does not comprise administering of an eIL-31 antigen, wherein said eIL-31 antigen comprises, or preferably is, a protein with the amino sequence selected from SEQ ID NO: 13 or a protein with an amino acid sequence of at least 90%, preferably of at least 92%, further preferably of at least 95%, and again further preferably of at least 98% amino acid sequence identity with SEQ ID NO:13.

In a further preferred embodiment, said method does not comprise co-administering of a composition comprising an eIL-31 antigen to said equine mammal, preferably to said horse. In another preferred embodiment, said method does not comprise co-administering of a composition comprising an eEotaxin antigen. In another preferred embodiment, said method does not comprise co-administering of a composition comprising an eIL-31 antigen or an eEotaxin antigen. In another preferred embodiment, said method does not comprise co-administering of a composition comprising an eIL-31 antigen and a composition comprising an eEotaxin antigen. In another preferred embodiment, said method does not comprise co-administering of a composition comprising an eIL-31 antigen, wherein said eIL-31 antigen comprises, or preferably is, a protein with the amino sequence selected from SEQ ID NO:13 or a protein with an amino acid sequence of at least 90%, preferably of at least 92%, further preferably of at least 95%, and again further preferably of at least 98% amino acid sequence identity with SEQ ID NO:13.

In a preferred embodiment, said method does not comprise administering of a composition comprising an eIL-31 antigen to said equine mammal, preferably to said horse, for at least eight weeks before and at least eight weeks after said administration of the inventive composition for use in said equine mammal, preferably in said horse, preferably for at least 3 months before and at least three months after said administration of the inventive composition for use in said equine mammal.

In a further preferred embodiment, said core particle is a virus-like particle (VLP), preferably a recombinant VLP. In again a further preferred embodiment, said VLP is derived from a plant virus or a bacteriophage, and wherein preferably said bacteriophage is a RNA bacteriophage. Thus, in a further preferred embodiment, said core particle is a virus-like particle (VLP), and wherein said VLP is derived from a RNA bacteriophage. Further preferred is a recombinant VLP of an RNA bacteriophage as core particle of the present invention. In a further preferred embodiment, said VLP comprises, consists essentially of, or alternatively consists of, recombinant coat proteins of an RNA bacteriophage, and wherein preferably said VLP comprises, consists essentially of, or alternatively consists of, recombinant coat proteins of RNA bacteriophage Qβ or of RNA bacteriophage AP205, and wherein further preferably said VLP comprises, consists essentially of, or alternatively consists of, recombinant coat proteins of RNA bacteriophage Qβ. In a further preferred embodiment, said VLP comprises, consists essentially of, or alternatively consists of, recombinant coat proteins comprising or preferably consisting of an amino acid sequence selected from (a) SEQ ID NO:14; (b) a mixture of SEQ ID NO:14 and SEQ ID NO:15; or (c) SEQ ID NO:16. In a further preferred embodiment, said VLP is a VLP of RNA bacteriophage Qβ. In a further preferred embodiment, said VLP comprises, consists essentially of, or alternatively consists of, recombinant coat proteins of RNA bacteriophage Qβ. Again in a further preferred embodiment, said VLP comprises, consists essentially of, or alternatively consists of, recombinant coat proteins comprising or preferably consisting of SEQ ID NO:14. In another preferred embodiment, said core particle is a virus-like particle (VLP) wherein said VLP is a VLP of RNA bacteriophage Qβ, and said VLP comprises, consists essentially of, or alternatively consists of, recombinant coat proteins of RNA bacteriophage Qβ, and wherein said recombinant coat proteins comprising or preferably consisting of SEQ ID NO:14.

In one embodiment, said VLP is not a VLP of an RNA bacteriophage, preferably said VLP is not a recombinant VLP of an RNA bacteriophage. In one embodiment, said virus-like particle is not a virus-like particle of an RNA-bacteriophage Qβ.

In a further preferred embodiment, said core particle is a virus-like particle (VLP), and wherein said VLP is derived from a plant virus. In another preferred embodiment, said VLP is a recombinant VLP, and wherein preferably said recombinant VLP is derived from a plant virus. In another preferred embodiment, said VLP is a VLP of cucumber mosaic virus (CMV).

In a preferred embodiment, said VLP is a modified VLP comprising, essentially consisting of, or alternatively consisting of, at least one modified VLP polypeptide, wherein said modified VLP polypeptide comprises, or preferably consists of, (a) a VLP polypeptide, and (b) a T helper cell epitope, wherein said VLP polypeptide comprises, or preferably consists of, (i) an amino acid sequence of a coat protein of a virus, preferably an amino acid sequence of a coat protein of a plant virus; or (ii) a mutated amino acid sequence, wherein the amino acid sequence to be mutated is an amino acid sequence of said coat protein of a virus, and wherein said mutated amino acid sequence and said coat protein of a virus show a sequence identity of at least 90%, preferably of at least 95%, further preferably of at least 98% and again more preferably of at least 99%.

In a preferred embodiment, said VLP is a modified VLP of cucumber mosaic virus (CMV), wherein said modified VLP of CMV comprises, essentially consists of, or alternatively consists of, at least one modified CMV polypeptide, wherein said modified CMV polypeptide comprises, or preferably consists of, (a) a CMV polypeptide, and (b) a T helper cell epitope; and wherein said CMV polypeptide comprises, or preferably consists of, (i) an amino acid sequence of a coat protein of CMV; or (ii) a mutated amino acid sequence, wherein the amino acid sequence to be mutated is an amino acid sequence of a coat protein of CMV, and wherein said mutated amino acid sequence and said coat protein of CMV show a sequence identity of at least 90%, preferably of at least 95%, further preferably of at least 98% and again more preferably of at least 99%.

In a preferred embodiment, said CMV polypeptide comprises, preferably consists of, an amino acid sequence of a coat protein of CMV. In another preferred embodiment, said CMV polypeptide comprises, preferably consists of a mutated amino acid sequence, wherein the amino acid sequence to be mutated is an amino acid sequence of a coat protein of CMV, and wherein said mutated amino acid sequence and said coat protein of CMV show a sequence identity of at least 90%, preferably of at least 95%, further preferably of at least 98% and again more preferably of at least 99%. Typically and preferably, said mutated amino acid sequence and said amino acid sequence to be mutated differ in least one and in at most 11, 10, 9, 8, 7, 6, 5, 4, 3, or 2 amino acid residues, and wherein preferably these differences are selected from (i) insertion, (ii) deletion, (iii) amino acid exchange, and (iv) any combination of (i) to (iii).

In another preferred embodiment, said CMV polypeptide comprises, or preferably consists of, (i) (a) an amino acid sequence of a coat protein of CMV, wherein said amino acid sequence comprises, or preferably consists of, SEQ ID NO:6 or (b) an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% of SEQ ID NO:6; or (ii) a mutated amino acid sequence, wherein said amino acid sequence to be mutated is said amino acid sequence as defined in (i) of this claim, and wherein said mutated amino acid sequence and said amino acid sequence to be mutated show a sequence identity of at least 95%, preferably of at least 98%, and more preferably of at least 99%.

In another preferred embodiment, said CMV polypeptide comprises, or preferably consists of, (a) an amino acid sequence of a coat protein of CMV, wherein said amino acid sequence comprises, or preferably consists of, SEQ ID NO:6 or (b) an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% of SEQ ID NO:6.

In another preferred embodiment, said CMV polypeptide comprises, or preferably consists of, (i) (a) an amino acid sequence of a coat protein of CMV, wherein said amino acid sequence comprises SEQ ID NO:17, or (b) an amino acid sequence of a coat protein of CMV comprising an amino acid sequence region, wherein said amino acid sequence region has a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:17; or (ii) a mutated amino acid sequence, wherein said amino acid sequence to be mutated is said amino acid sequence as defined in (i) of this claim, and wherein said mutated amino acid sequence and said amino acid sequence to be mutated show a sequence identity of at least 95%, preferably of at least 98%, and more preferably of at least 99%.

In a further preferred embodiment, said CMV polypeptide comprises, or preferably consists of, (a) an amino acid sequence of a coat protein of CMV, wherein said amino acid sequence comprises SEQ ID NO:17, or (b) an amino acid sequence of a coat protein of CMV comprising an amino acid sequence region, wherein said amino acid sequence region has a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:17.

In another preferred embodiment, said CMV polypeptide comprises, or preferably consists of, (i) (a) an amino acid sequence of a coat protein of CMV, wherein said amino acid sequence comprises, or preferably consists of, SEQ ID NO:6 or (b) an amino acid sequence having a sequence identity of at least 75%, preferably of at least 80%, more preferably of at least 85%, again further preferably of at least 90%, again more preferably of at least 95%, still further preferably of at least 98% and still again further more preferably of at least 99% of SEQ ID NO:6; and wherein said amino sequence as defined in (a) or (b) comprises SEQ ID NO:17; or wherein said amino sequence as defined in (a) or (b) comprises an amino acid sequence region, wherein said amino acid sequence region has a sequence identity of at least 75%, preferably of at least 80%, more preferably of at least 85%, again further preferably of at least 90%, again more preferably of at least 95%, still further preferably of at least 98% and still again further more preferably of at least 99% with SEQ ID NO:17; or (ii) a mutated amino acid sequence, wherein said amino acid sequence to be mutated is said amino acid sequence as defined in (i) of this claim, and wherein said mutated amino acid sequence and said amino acid sequence to be mutated show a sequence identity of at least 98% preferably of at least 99%.

In another preferred embodiment, said CMV polypeptide comprises, or preferably consists of, (a) an amino acid sequence of a coat protein of CMV, wherein said amino acid sequence comprises, or preferably consists of, SEQ ID NO:6 or (b) an amino acid sequence having a sequence identity of at least 90% of SEQ ID NO:6; and wherein said amino sequence as defined in (a) or (b) in this claim comprises SEQ ID NO:17; or wherein said amino sequence as defined in (a) or (b) in this claim comprises an amino acid sequence region, wherein said amino acid sequence region has a sequence identity of at least 90% with SEQ ID NO:17.

In another preferred embodiment, said T helper cell epitope replaces a N-terminal region of said CMV polypeptide. In another preferred embodiment the number of amino acids of said N-terminal region replaced is equal to or lower than the number of amino acids of which said T helper cell epitope consists.

In a further very preferred embodiment, said T helper cell epitope replaces a N-terminal region of said CMV polypeptide, and wherein the number of amino acids of said N-terminal region replaced is equal to or lower than the number of amino acids of which said T helper cell epitope consists. Typically and preferably, said replaced N-terminal region of said CMV polypeptide consists of 5 to 15 consecutive amino acids, preferably of 9 to 14 consecutive amino acids, more preferably of 11 to 13 consecutive amino acids.

In a further very preferred embodiment, said N-terminal region of said CMV polypeptide corresponds to amino acids 2-12 of SEQ ID NO:6.

In another very preferred embodiment, said T helper cell epitope is a universal T helper cell epitope. In another preferred embodiment, said T helper cell epitope consists of at most 20 amino acids.

In a very preferred embodiment, said Th cell epitope is a PADRE sequence. In a further very referred embodiment, said Th cell epitope comprises, preferably consists of, the amino acid sequence of SEQ ID NO: 10. In another very preferred embodiment, said Th cell epitope is a PADRE sequence, and wherein said Th cell epitope comprises, preferably consists of, the amino acid sequence of SEQ ID NO: 10.

In another preferred embodiment, said T helper cell epitope is derived from a human vaccine. In a very preferred embodiment, said Th cell epitope is derived from tetanus toxin. In a further very referred embodiment, said Th cell epitope has, preferably consists of, the amino acid sequence of SEQ ID NO:9. In another very preferred embodiment, said Th cell epitope is derived from tetanus toxin, and wherein said Th cell epitope has, preferably consists of, the amino acid sequence of SEQ ID NO:9.

In a very preferred embodiment, said Th cell epitope is a PADRE sequence, and wherein said Th cell epitope comprises, preferably consists of, the amino acid sequence of SEQ ID NO: 10; or wherein said Th cell epitope is derived from tetanus toxin, and wherein said Th cell epitope has, preferably consists of, the amino acid sequence of SEQ ID NO:9.

In a very preferred embodiment, said CMV polypeptide comprises, or preferably consists of, an amino acid sequence of a coat protein of CMV, wherein said amino acid sequence comprises, or preferably consists of, SEQ ID NO:6 or an amino acid sequence having a sequence identity of at least 95% of SEQ ID NO:6; and wherein said amino sequence comprises SEQ ID NO:17, and wherein said T helper cell epitope replaces the N-terminal region of said CMV polypeptide, and wherein said replaced N-terminal region of said CMV polypeptide consists of 11 to 13 consecutive amino acids, preferably of 11 consecutive amino acids, and wherein further preferably said N-terminal region of said CMV polypeptide corresponds to amino acids 2-12 of SEQ ID NO:6.

In another very preferred embodiment, said modified CMV polypeptide comprises, an amino acid sequence of SEQ ID NO: 11. In another very preferred embodiment, said modified CMV polypeptide consists of an amino acid sequence of SEQ ID NO: 11. In another very preferred embodiment, said modified CMV polypeptide comprises an amino acid sequence of SEQ ID NO:12. In another very preferred embodiment, said modified CMV polypeptide consists of an amino acid sequence of SEQ ID NO:12.

In a very preferred embodiment, said first attachment site and said second attachment site are linked via at least one covalent non-peptide-bond. In another very preferred embodiment, said first attachment site comprises, or preferably is, an amino group, preferably an amino group of a lysine. In a further very preferred embodiment, said second attachment site comprises, or preferably is, a sulfhydryl group, preferably a sulfhydryl group of a cysteine.

In a very preferred embodiment, the at least one first attachment site is an amino group, preferably an amino group of a lysine residue and the at least one second attachment site is a sulfhydryl group, preferably a sulfhydryl group of a cysteine residue or a sulfhydryl group that has been chemically attached to the at least one antigen of the invention. In a further preferred embodiment only one of said second attachment sites associates with said first attachment site through at least one non-peptide covalent bond leading to a single and uniform type of binding of said antigen to said modified virus-like particle, wherein said only one second attachment site that associates with said first attachment site is a sulfhydryl group, and wherein said antigen and said modified virus-like particle interact through said association to form an ordered and repetitive antigen array.

In one preferred embodiment of the invention, the antigen is linked to the modified VLP by way of chemical cross-linking, typically and preferably by using a heterobifunctional cross-linker. In preferred embodiments, the hetero-bifunctional cross-linker contains a functional group which can react with the preferred first attachment sites, preferably with the amino group, more preferably with the amino groups of lysine residue(s) of the modified VLP, and a further functional group which can react with the preferred second attachment site, i.e. a sulfhydryl group, preferably of cysteine(s) residue inherent of, or artificially added to the antigen, and optionally also made available for reaction by reduction. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, Sulfo-KMUS SVSB, SIA, and other cross-linkers available for example from the Pierce Chemical Company, and having one functional group reactive towards amino groups and one functional group reactive towards sulfhydryl groups. The above mentioned cross-linkers all lead to formation of an amide bond after reaction with the amino group and a thioether linkage with the sulfhydryl groups. Another class of cross-linkers suitable in the practice of the invention is characterized by the introduction of a disulfide linkage between the antigen and the modified VLP upon coupling. Preferred cross-linkers belonging to this class include, for example, SPDP and Sulfo-LC-SPDP (Pierce).

Linking of the antigen to the modified VLP by using a hetero-bifunctional cross-linker according to the preferred methods described above, allows coupling of the antigen to the modified VLP in an oriented fashion. Other methods of linking the antigen to the modified VLP include methods wherein the antigen is cross-linked to the modified VLP, using the carbodiimide EDC, and NHS. The antigen may also be first thiolated through reaction, for example with SATA, SATP or iminothiolane. The antigen, after deprotection if required, may then be coupled to the modified VLP as follows. After separation of the excess thiolation reagent, the antigen is reacted with the modified VLP, previously activated with a hetero-bifunctional cross-linker comprising a cysteine reactive moiety, and therefore displaying at least one or several functional groups reactive towards cysteine residues, to which the thiolated antigen can react, such as described above. Optionally, low amounts of a reducing agent are included in the reaction mixture. In further methods, the antigen is attached to the modified VLP, using a homo-bifunctional cross-linker such as glutaraldehyde, DSG, BM[PEO]4, BS3, (Pierce) or other known homo-bifunctional cross-linkers with functional groups reactive towards amine groups or carboxyl groups of the modified VLP.

In very preferred embodiments of the invention, the antigen is linked via a cysteine residue, having been added to either the N-terminus or the C-terminus of, or a natural cysteine residue within the antigen, to lysine residues of the modified virus-like particle. In a preferred embodiment, the composition of the invention further comprises a linker, wherein said linker associates said antigen with said second attachment site, and wherein preferably said linker comprises or alternatively consists of said second attachment site.

In a further very preferred embodiment of the invention, said core particle is a virus-like particle (VLP), preferably a recombinant VLP and said eIL-5 antigen comprises, or preferably is, a protein with the amino sequence selected from SEQ ID NO:1 or a protein with an amino acid sequence of at least 90%, preferably of at least 92%, further preferably of at least 95%, and again further preferably of at least 98% amino acid sequence identity with SEQ ID NO:1. In a further very preferred embodiment of the invention, said core particle is a modified VLP, preferably a recombinant modified VLP, in accordance with the present invention and said eIL-5 antigen comprises, or preferably is, a protein with the amino sequence selected from SEQ ID NO:1 or a protein with an amino acid sequence of at least 90%, preferably of at least 92%, further preferably of at least 95%, and again further preferably of at least 98% amino acid sequence identity with SEQ ID NO:1. In again a very preferred embodiment of the invention, said eIL-5 antigen comprises, or preferably is, a protein with the amino sequence selected from SEQ ID NO:1 or a protein with an amino acid sequence of at least 95%, and preferably of at least 98% amino acid sequence identity with SEQ ID NO:1. In again a very preferred embodiment of the invention, said eIL-5 antigen comprises, or preferably is, a protein with the amino sequence selected from SEQ ID NO:1 or a protein with an amino acid sequence of at least 95%, and preferably of at least 98% amino acid sequence identity with SEQ ID NO:1. In again a very preferred embodiment of the invention, said modified CMV polypeptide comprises, preferably consists of, an amino acid sequence of SEQ ID NO:11 and said eIL-5 antigen comprises, or preferably is, protein with the amino sequence selected from SEQ ID NO:1, SEQ ID NO:2, SEQ ID NO:3, SEQ ID NO:4, SEQ ID NO:5. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:1. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:2. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:3. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:4. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:5.

In a further very preferred embodiment of the invention, said core particle is a VLP, preferably a recombinant VLP, wherein said VLP is a modified VLP of cucumber mosaic virus (CMV), wherein said modified VLP of CMV comprises, essentially consists of, or alternatively consists of, at least one modified CMV polypeptide, wherein said modified CMV polypeptide comprises, or preferably consists of (a) a CMV polypeptide, and (b) a T helper cell epitope; and wherein said CMV polypeptide comprises, or preferably consists of, (i) an amino acid sequence of a coat protein of CMV; or (ii) a mutated amino acid sequence, wherein the amino acid sequence to be mutated is an amino acid sequence of a coat protein of CMV, and wherein said mutated amino acid sequence and said coat protein of CMV show a sequence identity of at least 90%, preferably of at least 95%, further preferably of at least 98% and again more preferably of at least 99%, and wherein said eIL-5 antigen comprises, or preferably is, a protein with the amino sequence selected from SEQ ID NO:1 or a protein with an amino acid sequence of at least 90%, preferably of at least 92%, further preferably of at least 95%, and again further preferably of at least 98% amino acid sequence identity with SEQ ID NO:1. In again a very preferred embodiment of the invention, said eIL-5 antigen comprises, or preferably is, a protein with the amino sequence selected from SEQ ID NO:1 or a protein with an amino acid sequence of at least 95%, and preferably of at least 98% amino acid sequence identity with SEQ ID NO:1. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence selected from SEQ ID NO:1, SEQ ID NO:2, SEQ ID NO:3, SEQ ID NO:4, SEQ ID NO:5. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:1. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:2. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:3. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:4. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:5.

In a further very preferred embodiment of the invention, said core particle is a VLP, preferably a recombinant VLP, wherein said VLP is a modified VLP of cucumber mosaic virus (CMV), wherein said modified VLP of CMV comprises, essentially consists of, or alternatively consists of, at least one modified CMV polypeptide, and wherein said modified CMV polypeptide comprises, preferably consists of, an amino acid sequence of SEQ ID NO:11 and said eIL-5 antigen comprises, or preferably is, a protein with the amino sequence selected from SEQ ID NO:1 or a protein with an amino acid sequence of at least 90%, preferably of at least 92%, further preferably of at least 95%, and again further preferably of at least 98% amino acid sequence identity with SEQ ID NO:1. In again a very preferred embodiment of the invention, said eIL-5 antigen comprises, or preferably is, a protein with the amino sequence selected from SEQ ID NO:1 or a protein with an amino acid sequence of at least 95%, and preferably of at least 98% amino acid sequence identity with SEQ ID NO:1. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence selected from SEQ ID NO:1, SEQ ID NO:2, SEQ ID NO:3, SEQ ID NO:4, SEQ ID NO:5, and wherein said composition does not comprise an eIL-31 antigen, wherein said eIL-31 antigen comprises, or preferably is, a protein with the amino sequence selected from SEQ ID NO:13 or a protein with an amino acid sequence of at least 90%, preferably of at least 92%, further preferably of at least 95%, and again further preferably of at least 98% amino acid sequence identity with SEQ ID NO:13. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:1, and wherein said composition does not comprise an eIL-31 antigen, wherein said eIL-31 antigen comprises, or preferably is, a protein with the amino sequence selected from SEQ ID NO:13 or a protein with an amino acid sequence of at least 90%, preferably of at least 92%, further preferably of at least 95%, and again further preferably of at least 98% amino acid sequence identity with SEQ ID NO:13. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:2, and wherein said composition does not comprise an eIL-31 antigen, wherein said eIL-31 antigen comprises, or preferably is, a protein with the amino sequence selected from SEQ ID NO:13 or a protein with an amino acid sequence of at least 90%, preferably of at least 92%, further preferably of at least 95%, and again further preferably of at least 98% amino acid sequence identity with SEQ ID NO:13. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:3, and wherein said composition does not comprise an eIL-31 antigen, wherein said eIL-31 antigen comprises, or preferably is, a protein with the amino sequence selected from SEQ ID NO:13 or a protein with an amino acid sequence of at least 90%, preferably of at least 92%, further preferably of at least 95%, and again further preferably of at least 98% amino acid sequence identity with SEQ ID NO:13. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:4, and wherein said composition does not comprise an eIL-31 antigen, wherein said eIL-31 antigen comprises, or preferably is, a protein with the amino sequence selected from SEQ ID NO:13 or a protein with an amino acid sequence of at least 90%, preferably of at least 92%, further preferably of at least 95%, and again further preferably of at least 98% amino acid sequence identity with SEQ ID NO:13. In again a very preferred embodiment of the invention, said modified CMV polypeptide comprises, preferably consists of, an amino acid sequence of SEQ ID NO:11 and said eIL-5 antigen comprises, or preferably is, a protein with the amino sequence of SEQ ID NO:5, and wherein said composition does not comprise an eIL-31 antigen, wherein said eIL-31 antigen comprises, or preferably is, a protein with the amino sequence selected from SEQ ID NO:13 or a protein with an amino acid sequence of at least 90%, preferably of at least 92%, further preferably of at least 95%, and again further preferably of at least 98% amino acid sequence identity with SEQ ID NO:13.

In a further very preferred embodiment of the invention, said core particle is a VLP, preferably a recombinant VLP, wherein said VLP is a modified VLP of cucumber mosaic virus (CMV), wherein said modified VLP of CMV comprises, essentially consists of, or alternatively consists of, at least one modified CMV polypeptide, and wherein said modified CMV polypeptide comprises, preferably consists of, an amino acid sequence of SEQ ID NO:12 and said eIL-5 antigen comprises, or preferably is, a protein with the amino sequence selected from SEQ ID NO:1 or a protein with an amino acid sequence of at least 90%, preferably of at least 92%, further preferably of at least 95%, and again further preferably of at least 98% amino acid sequence identity with SEQ ID NO:1. In again a very preferred embodiment of the invention, said modified CMV polypeptide comprises, preferably consists of, an amino acid sequence of SEQ ID NO:12 and said eIL-5 antigen comprises, or preferably is, a protein with the amino sequence of SEQ ID NO:1. In again a very preferred embodiment of the invention, said modified CMV polypeptide comprises, preferably consists of, an amino acid sequence of SEQ ID NO: 12 and said eIL-5 antigen comprises, or preferably is, a protein with the amino sequence of SEQ ID NO:2. In again a very preferred embodiment of the invention, said modified CMV polypeptide comprises, preferably consists of, an amino acid sequence of SEQ ID NO:12 and said eIL-5 antigen comprises, or preferably is, a protein with the amino sequence of SEQ ID NO:3. In again a very preferred embodiment of the invention, said modified CMV polypeptide comprises, preferably consists of, an amino acid sequence of SEQ ID NO: 12 and said eIL-5 antigen comprises, or preferably is, a protein with the amino sequence of SEQ ID NO:4. In again a very preferred embodiment of the invention, said modified CMV polypeptide comprises, preferably consists of, an amino acid sequence of SEQ ID NO:12 and said eIL-5 antigen comprises, or preferably is, a protein with the amino sequence of SEQ ID NO:5. Preferably, said composition does not comprise an eIL-31 antigen, wherein said eIL-31 antigen comprises, or preferably is, a protein with the amino sequence selected from SEQ ID NO: 13 or a protein with an amino acid sequence of at least 90%, preferably of at least 92%, further preferably of at least 95%, and again further preferably of at least 98% amino acid sequence identity with SEQ ID NO:13.

In a further preferred embodiment, said administration of said composition reduces at least one parameter or symptom associated with said urticaria, preferably recurrent urticaria, as compared to said at least one parameter or symptom associated with said urticaria, preferably recurrent urticaria, before said administration. In again a further preferred embodiment, said at least one parameter or symptom associated with said urticaria, preferably recurrent urticaria, is the level or severity grade of the urticaria by area of hives, and wherein preferably said reduction of said level or severity grade of the urticaria by area of hives is determined by a urticaria activity scoring test, and wherein typically and preferably said urticaria activity scoring test is effected as described in Example 1.

### EXAMPLES

Preferred core particle used in the present invention are virus-like particles (VLPs), in particular recombinant VLPs. In one embodiment, the VLP is VLP of RNA bacteriophage Qβ comprising, preferably consisting of, recombinant coat proteins of RNA bacteriophage Qβ of SEQ ID NO:14. Such virus-like particles of RNA bacteriophages are disclosed in WO 02/056905, the disclosure of which is herewith incorporated by reference in its entirety. In particular Example 18 of WO 02/056905 contains a detailed description of the preparation of VLP particles of RNA bacteriophage Qβ. In a very preferred embodiment, the VLP is a VLP of cucumber mosaic virus (CMV), in particular, a modified VLP of CMV, wherein T helper cell epitopes replace N-terminal regions of the CMV polypeptide. In a very preferred embodiment, the VLP is CMVtt830 comprising modified CMV polypeptides of SEQ ID NO: 11 or CMV-Npadr comprising modified CMV polypeptides of SEQ ID NO:12, preferably CMVtt830 comprising modified CMV polypeptides of SEQ ID NO:11, as described herein and as disclosed in WO 2016/062720. In particular Examples 1 to 6 of WO 2016/062720 contain a detailed description of the preparation of VLP particles of modified CMV polypeptides of SEQ ID NO:11 and SEQ ID NO:12.

Very preferred compositions of the present invention and used in the below examples are the above-mentioned CMVtt830-VLPs to which equine Interleukin-5 (eIL-5) antigens have been covalently coupled. The preparation of these compositions has been described in WO2017/042212, the disclosure of which is herewith incorporated by reference in its entirety. Thus, the cloning, expression and purification of equine Interleukin-5 (eIL-5) have been described in Example 1 of WO2017/042212 and the coupling of eIL-5 antigens to different VLPs in Example 10 of WO2017/042212, the disclosure of which is specifically herewith incorporated by reference in its entirety.

### Example 1

### Urticaria Activity Score (UAS)

An Urticaria Activity Score (UAS) has been applied which examines the severity of the urticaria by area of hives and pruritus severity of affected skin of the horse. The score range from 0 to 3, wherein 0 corresponds to no urticaria, 1 corresponds to single body part with hives and mild pruritus, 2 corresponds to moderate and almost half of body parts with hives, and 3 corresponds to almost whole body parts with hives and severe pruritus. A similar test and score for determination of urticaria symptoms has been established for human urticaria (Zuberbier T, et al., Allergy (2014) 69: 868-887; Zuberbier T, et al., Allergy (2018) 73:1393-1414).

### Example 2

### Cloning, Expression and Purification of Equine Interleukin-5 (eIL-5)

A. Cloning of eIL-5-C-his and Expression as Inclusion Bodies in E. coli

The cloning, expression and purification of equine Interleukin-5 (eIL-5) was conducted as described in Example 1 of WO2017/042212. Thus, the DNA sequence encoding for mature eIL-5 (mature Interleukin-5, equus caballus; UniProt 002699) and fragments thereof were generated by gene-synthesis. SEQ ID NO:1. In addition a linker (GGC) was added C-terminally. This insert was flanked by 5′ NdeI and 3′ XhoI and was integrated into pET 42b (+), containing an octa His-tag (to facilitate purification) and stop codon in frame. The recombinant protein expressed in E. coli is termed eIL-5-C-His (SEQ ID NO:2). Analogously, SEQ ID NO:3, SEQ ID NO:4 and SEQ ID NO:5 have been prepared. SEQ ID NO:3, SEQ ID NO:4 and SEQ ID NO:5 comprising a linker (GGC) and (except SEQ ID NO:5) a His-tag C-terminally. SEQ ID NO:2, SEQ ID NO:3 and SEQ ID NO:4, in particular SEQ ID NO:2 and SEQ ID NO:3 are interchangeably termed herein as “eIL-5-C-His”. Furthermore, when it is referred to eIL-5-C-His within this example section and the described figures, one of these eIL-5-C-His recombinant proteins have been used, in various examples even more than one or all been used in repeated experiments. Very preferred used eIL-5-C-His are SEQ ID NO:2 and SEQ ID NO:3.

B. Purification and Refolding of eIL-5-C-his

The purification and refolding of eIL-5-C-His was conducted as described in Example 1 of WO2017/042212.

C. Structure of Recombinant Homodimer Enriched eIL-5-C-his

The proper refolding of recombinant eIL-5-C-His was confirmed as described in Example 1 of WO2017/042212.

### Example 3

### Coupling of eIL-5 Antigens to CMVtt830 VLP, Immunization of Horses and Demonstration of Efficacy in Recurrent Urticaria Prone Horses

A. Coupling of eIL5-C-his to VLP of Qβ

The coupling of eIL5-C-His to Qβ VLP comprising coat proteins of SEQ ID NO:14 was conducted as described in Example 10 of WO2017/042212.

B. Coupling of eIL5-C-his to CMVtt830 VLP

CMVtt830 VLP comprising modified CMV polypeptides of SEQ ID NO:11 was produced as described in Example 4 of WO 2016/062720 and reacted with a 10 fold molar excess of the heterobifunctional cross-linker succinimidyl-6(β-maleimidopropionamido)-hexanoate (SMPH) (Pierce). Unreacted crosslinker was removed by passage over a PD-10 desalting column (GE Healthcare). The recombinant, purified and refolded eIL-5-C-His was reduced for 1 h with an equimolar excess amount of tri(2-carboxyethyl)phosphine hydrochloride (TCEP) in PBS or 20 mM Na₂PO₄/2 mM EDTA, pH 7.5 to reduce the cysteine residue contained in the linker. The reduced eIL-5-C-His was then mixed with the derivatized CMVtt830 VLPs at a molar ratio of VLP monomer to eIL-5-C-His protein of 1:2 and co-incubated for 4 hours at 22° C. to allow cross-linking. Optionally, the reaction was dialysed 12 hours against PBS pH 7.4 or 20 mM Na₂PO₄/2 mM EDTA, pH 7.5 using a 300 kDa cut-off dialysis membrane or free uncoupled eIL-5-C-His was removed by either size exclusion chromatography or tangential flow filtration using 100 kDa MWCO.

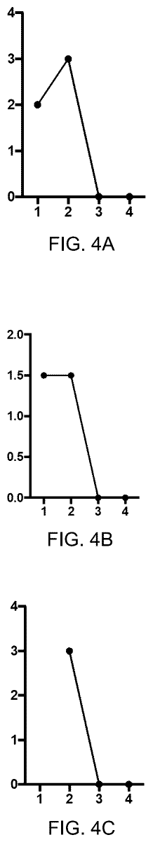

Analysis: Coomassie staining of SDS-PAGE (FIG. 4C): CMVtt830, eIL5-C-His, eIL5-C-His-CMVtt830 VLP were separated by SDS-PAGE. Subsequently gel was stained with Coomassie-Blue (0.025% Coomassie Brilliant BlueR-250, 40% methanol, 10% acetic acid) and de-stained with destainer (40% methanol, 10% acetic acid).

Western blot staining with anti-His antibody (FIG. 4D): CMV-tt830, eIL5-C-His, eIL5-C-His-CMVtt830 VLP were separated by SDS-PAGE and electroblotted onto a nitrocellulose membrane. The membrane was blocked for 1 h with 5% (w/v) BSA powder in PBST, then incubated with 10 ml of 1:1000 diluted anti-His antibody (monoclonal anti-His Tag antibody HRPO conjugate, Novagen Cat No. 71840) in 1% BSA (w/v) powder in PBST. The membrane was washed with PBST for 15 minutes and then developed with ECL (Amersham Pharmacia, Sweden) and exposed to Photographic film.

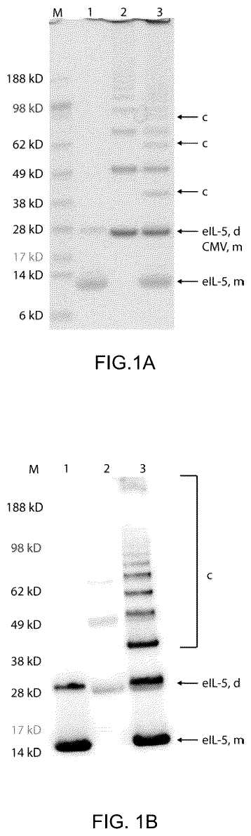

The covalent chemical coupling of eIL5-C-His to the CMVtt830 VLP was assessed by SDS-PAGE and Western blot analyses. Coomassie blue stained gels of the coupling reaction demonstrated the appearance of bands with molecular weights corresponding to those predicted for equine IL5-C-His covalently linked to CMV-tt830 (FIG. 1A). Furthermore, Western blot analyses showed co-localization of these bands when stained with anti-His antibody (FIG. 1B).

### C. Immunization Protocol

Horses, Placebo. Horses were injected subcutaneously on day 0, 28, and day 133 with 1,000 μl of PBS without presence of adjuvants. Horses were bled prior to immunization and at least either on day 56 and 84 of the immunization protocol and various additional time points post day 84. Sera were analyzed by ELISA.

Horses, eIL-5-C-His-Qβ VLP. In order to generate self-reactive antibodies to equine IL-5, horses were injected subcutaneously on day 0, 28, 56, and 84 with 300 μg of eIL5-C-His-Qβ VLP in 1,000 μl of PBS without presence of adjuvants. Horses were bled prior to immunization and at least on day 56, day 84 of the immunization protocol and various additional time points post day 84. Sera were analyzed by ELISA.

Horses, eIL-5-C-His-CMVtt830. In order to generate self-reactive antibodies to equine IL-5, in the first vaccination year, horses were injected subcutaneously on day 0, 28, and day 133 with 300 μg of eIL5-C-His-CMVtt830 VLP in 1,000 μl of sodium phosphate buffer without presence of adjuvants. Horses were bled prior to immunization and at least either on day 56 and 84 of the immunization protocol and various additional time points post day 84. Sera were analyzed by ELISA. In the second vaccination year, horses received a single booster vaccination after 1 year and 42 days with 300 μg of eIL5-C-His-CMVtt830 VLP in 1,000 μl of sodium phosphate buffer without presence of adjuvants. Horses were bled prior to booster injection and on day 56 of the immunization protocol and various additional time points post day 84.

### D. Sera Analysis by ELISA

Maxisorp 96 well ELISA plates (Nunc) were coated over night with 50 μl purified eIL-5-C-His, Qβ or purified CMVtt830 (5 μg/ml). Plates were washed 3 times with PBST blocked with Superblock (Thermo Scientific) in PBS for 2 hours at room temperature. Then plates were washed 3 times with PBST and three-fold dilutions of horse sera were added in Superblock (Thermo Scientific) in PBS and incubated at room temperature for 2 h. The plates were subsequently washed 3 times with PBST and incubated with anti-equine IgG conjugated with HRP (dilution 1:2000) at room temperature for 30 min. The plates were again washed 4 times with PBS and 50 μl/well developing solution (TMB) were added. After approximately 2 minutes of reaction at room temperature the ELISA was stopped with 25 μl per well 5% H₂SO₄. Absorbance was measured at 450 nm on a Tecan M200 spectrophotometer (Tecan, Austria).

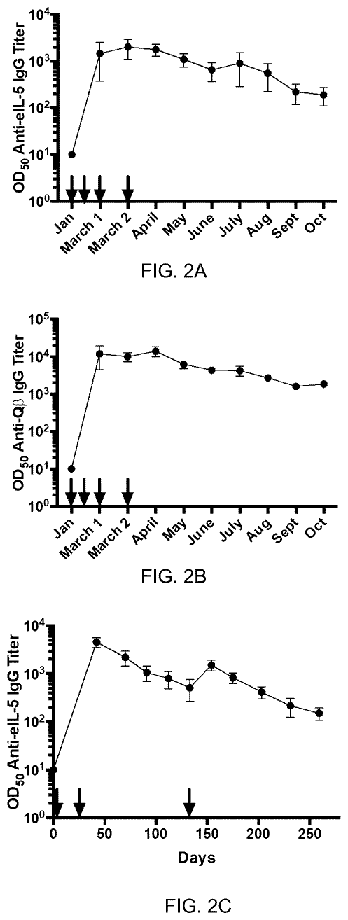

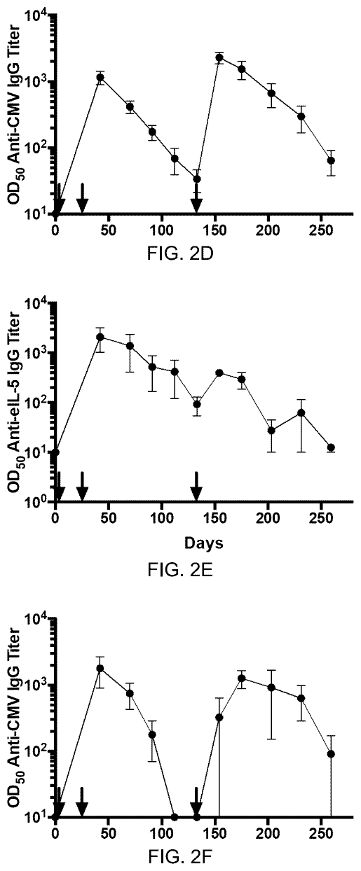

Pre-immune sera and various sera post immunization from horses vaccinated with eIL5-C-His-Qβ VLP were collected and analyzed by ELISA for antibodies against eIL-5-C-His (FIG. 2A) and antibodies against Qβ VLP (FIG. 2B). Pre-immune sera and various sera post immunization from eIL-5-C-His-CMVtt830 VLP vaccinated horses have been analyzed for antibodies against eIL-5-C-His (FIG. 2C, 2E) and antibodies against CMVtt830 (FIG. 2D, 2F). Horse sera were blotted as delta OD₅₀ (AOD₅₀) values, which were calculated from OD450 values for each dilution subtracted by corresponding naïve serum dilution. The result of vaccination in horses shows that immunological tolerance towards the self-antigen IL-5 was overcome. Half maximal titer anti-IL-5 at peak of response was in the range between 1:1,000-1:10,000.

### E. In Vivo Efficacy in Horses

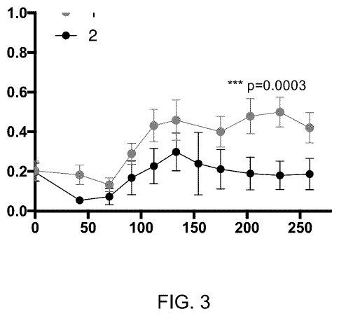

Eosinophil levels in blood were monitored in horses in two subsequent years, one placebo treated year and one treatment year using eIL-5-C-His-CMVtt830 vaccine and hence presence of anti-self eIL-5 antibodies. Moreover, three Icelandic horses with recurrent urticaria were scored by UAS (Urticaria Activity Score—Example 1) during four years: an untreated first year, a placebo treated second year, and a third and fourth year using eIL-5-C-His-CMVtt830 vaccination. Furthermore, antibody titers against eIL-5 and CMVtt830 were quantified, and eosinophil levels in blood were recorded for these three recurrent urticaria affected horses. Eosinophil levels in blood were monitored in thirteen horses in the placebo treated year (FIG. 3, line 1, grey) and treatment year using eIL-5-C-His-CMVtt830 vaccine (FIG. 3, line 2, black) showing statistically significant reduction of eosinophil levels in blood upon vaccination and presence of anti-eIL-5 antibody titer (FIG. 2C). Thus, the vaccine-induced anti-self eIL-5 antibodies in horses led to subsequent reduction of eosinophil levels in blood (FIG. 3).

In parallel, three horses (horse 1, horse 2, and horse 3) with yearly recurrent urticaria were vaccinated with eIL-5-C-His-CMVtt830 vaccine, and, as indicated, developed anti-eIL-5 antibodies (FIG. 2E) and anti-CMVtt830 antibodies (FIG. 2F) upon vaccination.

UAS (Urticaria Activity Score—see Example 1) was recorded in a first untreated year, a second placebo treated year, and the third and fourth eIL-5-C-His-CMVtt830 vaccinated year. All three horses developed urticaria hives in the untreated year and the placebo treated year (FIG. 4A, horse 1, FIG. 4B, horse 2, FIG. 4C, horse 3: row 1, untreated year, row 2, placebo treated year), whereas all three horses showed no clinical signs of urticaria in the third and fourth year vaccinated using eIL-5-C-His-CMVtt830 (FIG. 4A, horse 1, FIG. 4B, horse 2, FIG. 4C, horse 3: row 3, eIL-5-C-His-CMVtt830 vaccinated year, row 4, eIL-5-C-His-CMVtt830 vaccinated year).

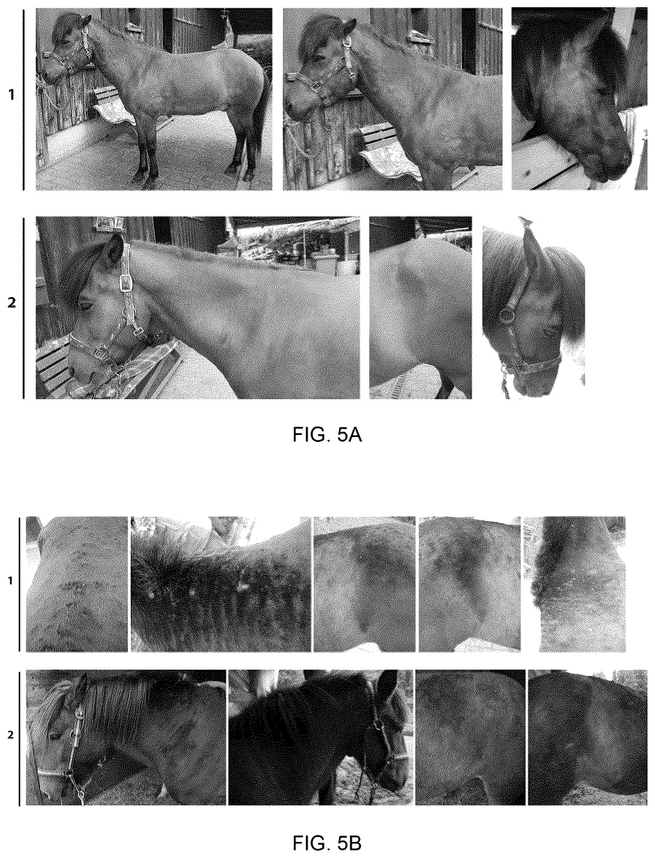

Moreover, photographs from horses 1 and 3 illustrated urticaria hives during the placebo treated year (FIG. 5A, horse 1, FIG. 5B, horse 3: lane 1), however, healthy skin from comparable time of the year when treated with eIL-5-C-His-CMVtt830 vaccine (FIG. 5A, horse 1, FIG. 5B, horse 3: lane 2).

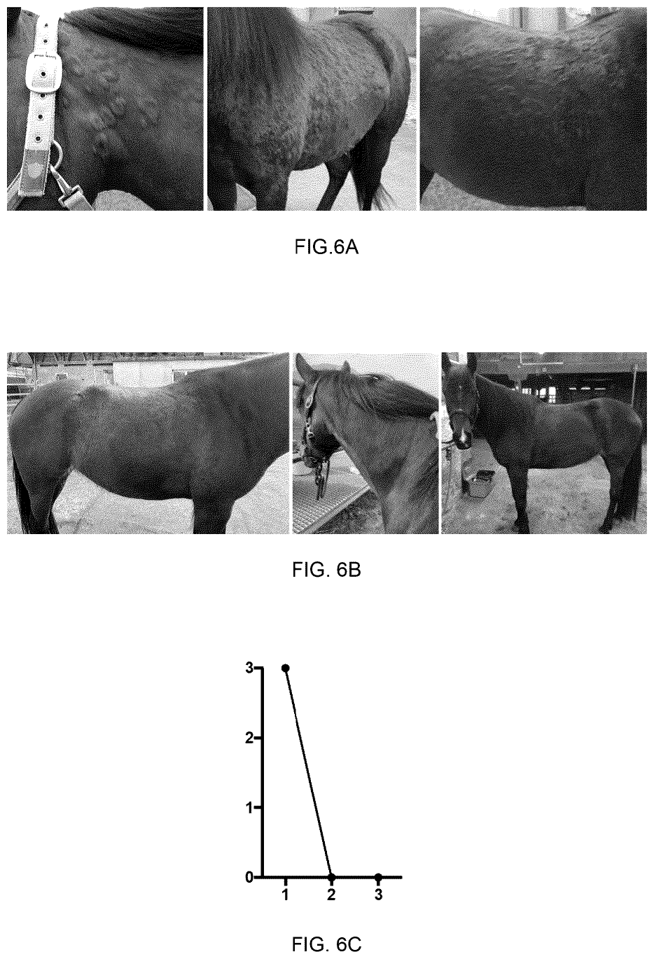

Another horse patient (*2011, Fell-pony/Appaloosa mix breed, horse 4) suffered for approximately two years almost non-intermittently, and notably during all four season of the year, from urticaria (FIG. 6A). Following the second vaccination onwards using eIL-5-C-His-CMVtt830, the horse was free of any clinical signs of urticaria (FIG. 6B). Before vaccination horse 4 showed a maximum UAS score of three (FIG. 6C, 1, UAS=3), and clinical signs disappeared upon two and three vaccinations (FIG. 6C, 2, UAS=0 and 3, UAS=0).

1. A method of prevention or treatment of urticaria of an equine mammal, wherein said method comprises administering an effective amount of a composition to said equine mammal, wherein said composition comprises:
(a) a core particle with at least one first attachment site; and
(b) at least one antigen with at least one second attachment site, wherein said at least one antigen is an equine Interleukin-5 antigen (eIL-5 antigen), wherein said eIL-5 antigen comprises a protein with the amino sequence selected from SEQ ID NO:1 or a protein with an amino acid sequence of at least 90% amino acid sequence identity with SEQ ID NO:1;
wherein (a) and (b) are linked through said at least one first and said at least one second attachment site via at least one non-peptide covalent bond.

2. The method according to claim 1, wherein said prevention or treatment of urticaria is not the prevention or treatment of insect bite hypersensitivity (IBH) of an equine mammal.

3. The method according to claim 1, wherein said prevention or treatment of urticaria is not the prevention or treatment of urticaria caused by insect bite hypersensitivity (IBH) of an equine mammal.

4. The method according to claim 1, wherein said composition does not comprise an equine Interleukin-31 antigen (eIL-31 antigen).

5. The method according to claim 1, wherein said method does not comprise administering of a composition comprising an eIL-31 antigen to said equine mammal.

6. The method according to claim 1, wherein said method does not comprise administering of an eIL-31 antigen to said equine mammal.

7. The method according to claim 1, wherein said eIL-5 antigen comprises a protein with the amino sequence selected from SEQ ID NO:1, SEQ ID NO:2, SEQ ID NO:3, SEQ ID NO:4 and SEQ ID NO:5.

8. The method according to claim 1, wherein said core particle is a virus-like particle (VLP).

9. The method according to claim 6, wherein said VLP is a modified VLP comprising at least one modified VLP polypeptide, wherein said modified VLP polypeptide comprises
(a) a VLP polypeptide, and
(b) a T helper cell epitope,
wherein said VLP polypeptide comprises
(i) an amino acid sequence of a coat protein of a virus; or
(ii) a mutated amino acid sequence, wherein the amino acid sequence to be mutated is an amino acid sequence of said coat protein of a virus, and wherein said mutated amino acid sequence and said coat protein of a virus show a sequence identity of at least 90%.

10. The method according to claim 6, wherein said VLP is a modified VLP of cucumber mosaic virus (CMV), wherein said modified VLP of CMV comprises, essentially consists of, or alternatively consists of, at least one modified CMV polypeptide, wherein said modified CMV polypeptide comprises
(a) a CMV polypeptide, and
(b) a T helper cell epitope; and
wherein said CMV polypeptide comprises
(ii) an amino acid sequence of a coat protein of CMV; or
(ii) a mutated amino acid sequence, wherein the amino acid sequence to be mutated is an amino acid sequence of a coat protein of CMV, and wherein said mutated amino acid sequence and said coat protein of CMV show a sequence identity of at least 90%.

11. The method according to claim 9, wherein said T helper cell epitope replaces a N-terminal region of said CMV polypeptide, and wherein said N-terminal region of said CMV polypeptide corresponds to amino acids 2-12 of SEQ ID NO:6.

12. The method according to claim 9, wherein said CMV polypeptide comprises, an amino acid sequence of a coat protein of CMV, wherein said amino acid sequence comprises SEQ ID NO:6 or an amino acid sequence having a sequence identity of at least 95% of SEQ ID NO:6; and wherein said amino sequence comprises SEQ ID NO:17.

13. The method according to claim 11, wherein said T helper cell epitope replaces the N-terminal region of said CMV polypeptide, and wherein said replaced N-terminal region of said CMV polypeptide consists of 11 to 13 consecutive amino acids.

14. The method according to claim 9, wherein said modified CMV polypeptide comprises an amino acid sequence of SEQ ID NO:11 or SEQ ID NO:12.

15. The method according to claim 1, wherein said administration of said composition reduces at least one parameter or symptom associated with said urticaria, as compared to said at least one parameter or symptom associated with said urticaria before said administration.

16. The method of claim 1, wherein said urticarial is recurrent urticaria.

17. The method of claim 1, wherein said equine mammal is a horse.

18. The method of claim 8, wherein said virus-like particle (VLP) is a recombinant VLP.

19. The method of claim 15, wherein said at least one parameter or symptom associated with said urticaria is the level or severity grade of the urticaria by area of hives.

20. The method of claim 19, wherein said reduction of said level or severity grade of the urticaria by area of hives is determined by a urticaria activity scoring test.


SEQUENCE LISTING (17 sequences)

SEQ ID NO: 1 - protein, 115 aa, synthetic construct
  REGION 1..115: note = eIL5 mature
LAVESPMNRLVAETLTLLSTHRTLLIGDGNLMIPTPEHKNHQLCIEEVFQGIDTLKNQTVQGDAVAKLFQNLSLIKGYIDLQKKKCGGERWRVKQFLDYLQEFLGVINTEWTIEG

SEQ ID NO: 2 - protein, 127 aa, synthetic construct
  REGION 1..127: note = eIL-5-C-His1
AVESPMNRLVAETLTLLSTHRTLLIGDGNLMIPTPEHKNHQLCIEEVFQGIDTLKNQTVQGDAVAKLFQNLSLIKGYIDLQKKKCGGERWRVKQFLDYLQEFLGVINTEWTIEGGGCLEHHHHHHHH

SEQ ID NO: 3 - protein, 124 aa, synthetic construct
  REGION 1..124: note = eIL-5-C-His2
LAVESPMNRLVAETLTLLSTHRTLLIGDGNLMIPTPEHKNHQLCIEEVFQGIDTLKNQTVQGDAVAKLFQNLSLIKGYIDLQKKKCGGERWRVKQFLDYLQEFLGVINTEWTIEGGGCHHHHHH

SEQ ID NO: 4 - protein, 124 aa, synthetic construct
  REGION 1..124: note = eIL5 antigen var1
GGCLAVESPMNRLVAETLTLLSTHRTLLIGDGNLMIPTPEHKNHQLCIEEVFQGIDTLKNQTVQGDAVAKLFQNLSLIKGYIDLQKKKCGGERWRVKQFLDYLQEFLGVINTEWTIEGHHHHHH

SEQ ID NO: 5 - protein, 118 aa, synthetic construct
  REGION 1..118: note = eIL5 antigen var2
GGCLAVESPMNRLVAETLTLLSTHRTLLIGDGNLMIPTPEHKNHQLCIEEVFQGIDTLKNQTVQGDAVAKLFQNLSLIKGYIDLQKKKCGGERWRVKQFLDYLQEFLGVINTEWTIEG

SEQ ID NO: 6 - protein, 218 aa, Cucumber mosaic virus
MDKSESTSAGRSRRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 7 - protein, 217 aa, Cucumber mosaic virus
DKSESTSAGRNRRRRPRRGSRSASSSADANFRVLSQQLSRLNKTLAAGRPTINHPTFVGSERCKPGYTFSSITLKPPKIDRGSYYGKRLLLPDSVTEFDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDIEHQRIPTSGVLPV

SEQ ID NO: 8 - protein, 218 aa, Cucumber mosaic virus
MDKSESPNASRTSRRRRPRRGSRSASGADAGLRALTQQMLKLNKTLAIGRPTLNHPTFVGSASCKPGYTFTSITLKPPEIEKGSYFGRRLSLPDSVTDYDKKLVSRIQIRINPLPKFDSTVWVTVRKVPSSSDLSVATISAMFGDGNSPVLVYQYTASGVQANNKLLYDLSEMRADIGDMRKYAVLVYSKDDKLEEDEIVLHVDVEHQRIPISRMLPT

SEQ ID NO: 9 - protein, 14 aa, synthetic construct
  REGION 1..14: note = tetanus toxoid epitope tt830
QYIKANSKFIGITE

SEQ ID NO: 10 - protein, 13 aa, synthetic construct
  REGION 1..13: note = PADRE
AKFVAAWTLKAAA

SEQ ID NO: 11 - protein, 222 aa, synthetic construct
  REGION 1..222: note = CMV-Ntt830
MGQYIKANSKFIGITERRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 12 - protein, 220 aa, synthetic construct
  REGION 1..220: note = CMV-Npadr
MAKFVAAWTLKAAARRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 13 - protein, 123 aa, synthetic construct
  REGION 1..123: note = eIL-31 mature
GPIYQLQPKEIQAIIVELQNLSKKLLDDYLNKEKGVQKFDSDLPSCFTSDSQAPGNINSSAILPYFKAISPSLNNDKSLYIIEQLDKLNFQNAPETEVSMPTDNFERKRFILTILRWFSNCLE

SEQ ID NO: 14 - protein, 132 aa, unidentified
  REGION 1..132: note = Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 15 - protein, 329 aa, unidentified
  REGION 1..329: note = Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 16 - protein, 131 aa, unidentified
  REGION 1..131: note = Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 17 - protein, 27 aa, synthetic construct
  REGION 1..27: note = aa 2-28 of SEQ ID NO:24
DKSESTSAGRSRRRRPRRGSRSAPSSA